(19) United States
(12) Patent Application Publication
(10) Pub. No.: US 2012/0136221 A1
(43) Pub. Date: May 31, 2012

(54) System and method for monitoring the health of a hospital patient

(76) Inventors: Roger KILLEN, Exeter (GB); Roy Margolis, London (GB)

(21) Appl. No.: 13/373,140

(22) Filed: Nov. 4, 2011

(30) Foreign Application Priority Data
Nov. 5, 2010 (GB) 1018774.8
Nov. 30, 2010 (GB) 1020261.2

(51) Int. Cl. A61B 5/00

(52) U.S. Cl. 600/300

(57) ABSTRACT

A system for monitoring the health of a hospital patient. The system includes a score engine configured to identify the status of a patient's health using one or more measurements relating to the patient, and a list manager configured to identify a medical practitioner associated with the patient. The system is configured to send a message to the medical practitioner in dependence on the identified status of the patient's health. A corresponding method is also provided.

### CROSS-REFERENCE(S) TO RELATED APPLICATION(S)

This application is related to, and claims a benefit of priority under one or more of 35 U.S.C. 119(a)-119(d) from co-pending foreign patent application 1018774.8, filed in the United Kingdom on Nov. 5, 2010 and co-pending foreign patent application 1020261.2 filed in the United Kingdom on Nov. 30, 2010 under the Paris Convention, the entire contents of both of which are hereby expressly incorporated herein by reference for all purposes.

### TECHNICAL FIELD

The present invention relates to hospital patient monitoring. More particularly, the present invention relates to a system and method for monitoring the health of a hospital patient.

### BACKGROUND

It is a responsibility of a hospital to monitor the health of each patient admitted. Usually, a doctor is assigned responsibility of one or a number of patients. If a patient's health begins to deteriorate then the doctor assigned to that patient should be contacted so that they can administer treatment to the patient. Accordingly, there is a need to monitor the health of each hospital patient so that the deteriorating health of any patient can be detected. Furthermore, once the deteriorating health of a patient has been detected, there is a need to notify the doctor responsible for that patient.

It is known to monitor a set of vital signs for each hospital patient. The set of vital signs may include: pulse, respiratory rate, blood pressure and temperature. Periodically, a hospital employee, such as a nurse, measures each vital sign in the set and records a corresponding value for each vital sign in a chart hung on the patient's bed. The measured set of vital signs can be used by the hospital employee to determine if the health of the patient is deteriorating at a particular point in time. Additionally, the hospital employee may use the measured set of vital signs to calculate a score or indicator which can be used to determine if the health of the patient is deteriorating. The score or indicator may be an Early Warning Score (EWS). The calculated score or indicator can be used by the hospital employee to determine if the health of the patient is deteriorating at a particular point in time. In the event that the hospital employee determines that the patient's health is deteriorating, the hospital employee can contact the doctor responsible for the patient to notify him that their patient requires treatment. The chart hung on a patient's bed can indicate which doctor is responsible for the patient.

There are many problems with the above-described mechanism for, detecting the deteriorating health of a patient, and contacting the doctor responsible in the event of detection. Firstly, clerical errors may be introduced when calculating the EWS since the hospital employee must perform a complex mathematical operation. Secondly, there is only one up-to-date record of the patients vital signs, and this is with the patient, therefore, the doctor must travel to the patient before he can identify why the patient's health is deteriorating. Thirdly, multiple doctors can be responsible for a single patient and so, when the patient's health deteriorates, it can be difficult to know which doctor to contact first. Additionally, time can be wasted when multiple doctors rush to attend to the same patient at the same time.

### SUMMARY OF THE INVENTION

From a first aspect there is provided a system for monitoring the health of a hospital patient, the system comprising: a score engine configured to identify the status of a patient's health using one or more measurements relating to the patient; and, a list manager configured to identify a medical practitioner associated with the patient; wherein the system is configured to send a message to the medical practitioner in dependence on the identified status of the patient's health.

The system may further comprise a remote device being a mobile computing device configured to receive the sent message and notify a user of the remote device of message receipt.

The system may further comprise: a measuring device being a mobile computing device configured to receive an input from a user of the device, the input comprising the one or more measurements, the measuring device being further configured to transmit the input; and, wherein the system is arranged so that the score engine receives the one or more measurements from the input transmitted by the measuring device.

### Server

The system may further comprise a server for storing patient information relating to the patient such that it is retrievable, the patient information including the one or more measurements relating to the patient; and, wherein the system is arranged so that the server receives the input transmitted by the measuring device.

In one embodiment the patient information may include administrative information relating to the patient.

### Score Engine

In one embodiment the score engine calculates a score indicating the status of the patient's health using a risk algorithm which uses at least one of the one or more measurements. The one or more measurements may include physiological measurements, and the physiological measurements may include at least one of the following: pulse, temperature, blood pressure, oxygen saturation, respiration rate, neurological indicator, inspired oxygen.

In one embodiment the risk algorithm has an AUROC value greater than or equal to 0.80, and preferably has an AUROC value of greater than or equal to 0.88. In one embodiment the risk algorithm is the VitalPAC early warning score (ViEWS).

In one embodiment the score engine stores a predefined threshold, and the system sends the message when the value of the score crosses the predefined threshold.

Alternatively the score engine stores multiple predefined thresholds, and the system sends the message when the value of the score crosses at least one of the predefined thresholds.

Alternatively, the score engine stores multiple predefined thresholds, and the system sends the message when the value of the score crosses each of the predefined thresholds.

In one embodiment the system sends the message when the value of the score crosses a threshold in only one direction.

### List Manager

In one embodiment the list manager stores an association between the patient and the medical practitioner, and wherein the list manager identifies the medical practitioner by identifying the association.

In one embodiment the list manager stores a patient list for the medical practitioner, the patient list including patients associated with the medical practitioner, wherein the list manager identifies the patient in the patient list to identify that the medical practitioner is associated with the patient.

More specifically the list manager may store patient lists for one or more medical practitioners, each patient list including patients associated with a medical practitioner corresponding to the patient list, wherein the list manager identifies a medical practitioner associated with the patient by identifying a patient list containing the patient.

In one embodiment only patient lists for medical practitioners on duty are identified.

In one embodiment when the list manager identifies that the patient is in a single patient list, the list manager identifies the medical practitioner corresponding to the single patient list, and the system sends the message to the identified medial practitioner.

In one embodiment when the list manager identifies that the patient is in multiple patient lists, the list manager identifies each medical practitioner corresponding to each list, and the system sends the message to at least one identified medical practitioner.

In one embodiment when the list manager identifies that the patient is in multiple patient lists, the list manager identifies each medical practitioner corresponding to each list, and the system sends the message to each identified medical practitioner.

In one embodiment the score engine stores multiple predefined thresholds, and the system sends the message to at least one of the identified medical practitioners each time the value of the score crosses one of the thresholds.

In one embodiment the system sends the message to a different one of the identified medical practitioners when the value of the score crosses each different threshold.

In one embodiment the system sends the message to a different one of the identified medical practitioners in dependence on the value of the score.

One embodiment further comprises an interface for allowing a user of the system to access the list manager, the interface having a display and being capable of displaying and modifying patient list information.

### Message

In one embodiment content of the message indicates the score value which crossed the threshold and the patient whose measurements were used in calculating the score value.

For example, in one embodiment the message content indicates the direction which the score crossed the threshold.

In another embodiment the message content changes in dependence on the score threshold value.

In one embodiment the message indicates patient information. For example, the patient information may include at least one of the following: patient medical records, patient test results, patient details, patient location.

### Remote Device

In one embodiment the remote device receives messages sent to the current user of the remote device, wherein the current user is identified as the user logged into the remote device.

In one embodiment the remote device is a notification device configured to provide the user with instructions for obtaining content of the message. The notification device may be a pager or a beeper.

In one embodiment the remote device is an analysis device configured to provide the user with the message content. The analysis device may be additionally configured to provide the user with patient information, to assist the user in administering the correct treatment to the patient. In such a case the patient information may include at least one of the following: patient medical records, patient test results, patient details, patient location.

In one embodiment the analysis device is additionally configured to receive data from the current user and transmit the data. The server may then be configured to receive the data transmitted from the analysis device, the data comprising updated patient information for updating or creating patient information on the server.

In one embodiment the list manager is configured to receive the data transmitted from the analysis device, the data comprising updated patient association information for updating or creating a patient list and/or patient association relating to the current user on the list manager.

In one embodiment the data indicates whether or not the current user of the analysis device is going to take responsibility for progressing the care of the patient, the system being further configured to deliver said data to a measuring device or an analysis device.

### Measuring Device

In one embodiment the measuring device is configured to receive the input from the user via a graphical user interface of the measuring device.

In one embodiment the input additionally comprises administrative information relating to the patient. The administrative information may include at least one of the following: a patient location, a medical practitioner associated with the patient.

In one embodiment the measuring device notifies a current user of the measuring device when the one or more measurements need to be taken from the patient, or how long overdue are those one or more measurements, wherein the measuring device identifies the current user as the user logged-in to the measuring device.

In one embodiment the system is configured so that the measuring device receives a notification in response to transmitting the input, the notification indicating whether or not someone has taken responsibility for progressing the care of the patient. In particular the system may be configured so that the notification is received by the measuring device a predetermined time after the input is transmitted from the measuring device.

### General

In one embodiment the system may further comprise a base-station configured to communicate wirelessly with the remote device and the measuring device, the base-station including at least one of the score engine and the list manager. The base-station may further include the server.

In one embodiment the system may further comprise a base-station configured to communicate wirelessly with the remote device and the measuring device, the base-station including the list manager, and wherein the measuring device includes the score engine. The base-station may further include the server.

In one embodiment identifying the status of the patient's health comprises identifying changing health of the patient; and, sending a message to the medical practitioner in dependence on the identified status of the patient's health comprises sending a message to the medical practitioner if changing health of the patient is identified. Herein, changing health may comprise deteriorating health.

### Method

From another aspect there is provided a computer implemented method for monitoring the health of a hospital patient, the method comprising: electronically identifying the status of a patient's health, using one or more measurements relating to the patient; and, electronically identifying a medical practitioner associated with the patient and sending an electronic message to the medical practitioner, in dependence on the identified status of the patient's health.

In one embodiment the method may further comprise receiving the sent electronic message at a remote device, the remote device being a mobile computing device; and, notifying the medical practitioner of message receipt, the medical practitioner being a user of the remote device.

One embodiment may further comprise electronically receiving the one or more measurements at a measuring device, the measuring device being a mobile computing device.

### Server

One embodiment may further comprise electronically storing patient information relating to the patient such that it is retrievable, the patient information including the one or more measurements relating to the patient. Here, the patient information may include administrative information relating to the patient, and the method may further include electronically receiving the administrative information at the measuring device with the one or more measurements.

### Score Engine

One embodiment may further comprise electronically calculating a score indicating the status of the patient's health using a risk algorithm which uses at least one of the one or more measurements. Here; the one or more measurements may include physiological measurements, and the physiological measurements may include at least one of the following: pulse, temperature, blood pressure, oxygen saturation, respiration rate, neurological indicator, inspired oxygen.

In one embodiment the risk algorithm has an AUROC value greater than or equal to 0.80, and preferably has an AUROC value of greater than or equal to 0.88.

In one embodiment the risk algorithm is the VitalPAC early warning score (VIEWS).

One embodiment may further comprise electronically storing a predefined threshold; and sending the electronic message when the value of the score crosses the predefined threshold.

One embodiment may further comprise electronically storing multiple predefined thresholds; and sending the electronic message when the value of the score crosses at least one of the predefined thresholds.

One embodiment may further comprise electronically storing multiple predefined thresholds; and sending the electronic message when the value of the score crosses each of the predefined thresholds.

In the above, the electronic message may be sent when the value of the score crosses a threshold in only one direction.

### List Manager

One embodiment may further comprise electronically storing an association between the patient and the medical practitioner, wherein electronically identifying a medical practitioner associated with the patient comprises identifying the electronically stored association.

One embodiment may further comprise electronically storing a patient list for the medical practitioner, the patient list including patients under the responsibility of the medical practitioner; wherein electronically identifying a medical practitioner associated with the patient comprises electronically identifying the patient in the patient list.

One embodiment may further comprise electronically storing patient lists for one or more medical practitioners, each patient list including patients under the responsibility of a medical practitioner corresponding to the patient list; wherein electronically identifying a medical practitioner associated with the patient comprises electronically identifying a patient list containing the patient.

In one embodiment only patient lists for medical practitioners on duty are electronically identified.

One embodiment may further comprise when it is electronically identified that the patient is in a single patient list, electronically identifying the medical practitioner corresponding to the single patient list, and sending the electronic message to the identified medial practitioner.

One embodiment may further comprise when it is electronically identified that the patient is in multiple patient lists, electronically identifying each medical practitioner corresponding to each list, and sending the electronic message to at least one identified medical practitioner.

One embodiment may further comprise when it is electronically identified that the patient is in multiple patient lists, electronically identifying each medical practitioner corresponding to each list, and sending the electronic message to each identified medical practitioner.

One embodiment may further comprise electronically storing multiple predefined thresholds; and sending the electronic message to at least one of the identified medical practitioners each time the value of the score crosses one of the thresholds.

One embodiment may further comprise sending the electronic message to a different one of the identified medical practitioners when the value of the score crosses each different threshold.

One embodiment may further comprise sending the electronic message to a different one of the identified medical practitioners in dependence on the value of the score.

### Message

One embodiment may further comprise setting the content of the electronic message to indicate the score value which crossed the threshold and the patient whose measurements were used in calculating the score value.

One embodiment may further comprise setting the content of the electronic message to indicate the direction which the score crossed the threshold.

One embodiment may further comprise changing the content of the electronic message in dependence on the score threshold value.

One embodiment may further comprise setting the content of the electronic message to indicate patient information.

In one embodiment the patient information includes at least one of the following: patient medical records, patient test results, patient details, patient location.

### Remote Device

One embodiment may further comprise providing the medical practitioner with electronic instructions for obtaining content of the electronic message, at the remote device.

In one embodiment the electronic instructions include: a telephone number, a patient identifier, a patient location.

One embodiment may further comprise providing the medical practitioner with the content of the electronic message, at the remote device.

One embodiment may further comprise providing the medical practitioner with electronic patient information, at the remote device, to assist the medical practitioner in administering the correct treatment to the patient.

In one embodiment the patient information includes at least one of the following: patient medical records, patient test results, patient details, patient location.

One embodiment may further comprise electronically receiving an input from the medical practitioner, at the remote device.

In one embodiment the input comprises updated patient information and/or updated patient association information to be stored electronically.

In one embodiment the input comprises an indication of whether or not the medical practitioner is going to take responsibility for progressing the care of the patient, the input being for delivery to a measuring device or a remote device.

### Measuring Device

One embodiment may further comprise electronically receiving an input from a user, at the measuring device, the input comprising the one or more measurements relating to the patient.

In one embodiment the input additionally comprises administrative information relating to the patient.

In one embodiment the administrative information includes at least one of the following: a patient location, a medical practitioner associated with the patient.

One embodiment may further comprise electronically notifying the user when the one or more measurements need to be taken from the patient, or how long overdue are those one or more measurements.

One embodiment may further comprise electronically receiving, at the measuring device, an indication of whether or not someone has taken responsibility for progressing the care of the patient.

In one embodiment the indication is electronically received at the measuring device a predetermined time after the measuring device receives the one or more measurements.

### General

One embodiment, may further comprise electronically identifying the status of the patient's health comprises electronically identifying changing health of the patient; and, electronically identifying a medical practitioner associated with the patient and sending an electronic message to the medical practitioner, in dependence on the identified status of the patient's health comprises electronically identifying a medical practitioner associated with the patient and sending an electronic message to the medical practitioner, if changing health of the patient is identified.

In one embodiment changing health comprises deteriorating health.

Another aspect may further present a system for monitoring the health of a hospital patient, the system comprising: means for electronically identifying the status of a patient's health using one or more measurements relating to the patient; means for electronically identifying a medical practitioner associated with the patient; and, means for sending an electronic message to the medical practitioner in dependence on the identified health status of the patient.

One embodiment may further comprise means for electronically receiving the one or more measurements relating to the patient.

One embodiment may further comprise means for electronically receiving the sent message; and means for electronically notifying the medical practitioner of message receipt.

In one embodiment a base-station provides at least one of the following: the means for electronically identifying the status of a patient's health using one or more measurements relating to the patient; the means for electronically identifying a medical practitioner associated with the patient; and the means for sending an electronic message to the medical practitioner in dependence on the health status of the patient.

In one embodiment a first mobile computing device provides the means for electronically receiving the one or more measurements relating to the patient.

In one embodiment a second mobile computing device provides at least one of the following: the means for electronically receiving the sent message; and the means for electronically notifying the medical practitioner of message receipt.

In one embodiment identifying the status of the patient's health comprises identifying changing health of the patient; and means for sending an electronic message to the medical practitioner in dependence on the identified health status of the patient comprises means for sending an electronic message to the medical practitioner if changing health of the patient is identified.

### BRIEF DESCRIPTION OF THE DRAWINGS

Various example embodiments of the present invention will now be described with reference to the following drawings, wherein like reference numerals relate to like components, and wherein:—

FIG. 1 is a diagram of a system according to a first embodiment of the present invention;

FIG. 2 is a diagram of a mobile computing device according to the first embodiment;

FIG. 3 is a flow diagram illustrating the operation of a measuring device of the first embodiment;

FIG. 4 is a flow diagram illustrating the operation of an analysis device of the first embodiment;

FIGS. 5 and 6 are tables illustrating the operation of the score engine of the first embodiment;

FIG. 7 is a diagram of an analysis device according to a second embodiment of the present invention;

FIG. 8 is a diagram of an analysis device according to a third embodiment of the present invention; and,

FIGS. A1 to A75 are screen shots from an analysis device operated in accordance with software as defined by the functional requirements of Annex A.

### DESCRIPTION OF EMBODIMENTS OF THE INVENTION

### Overview of System Architecture

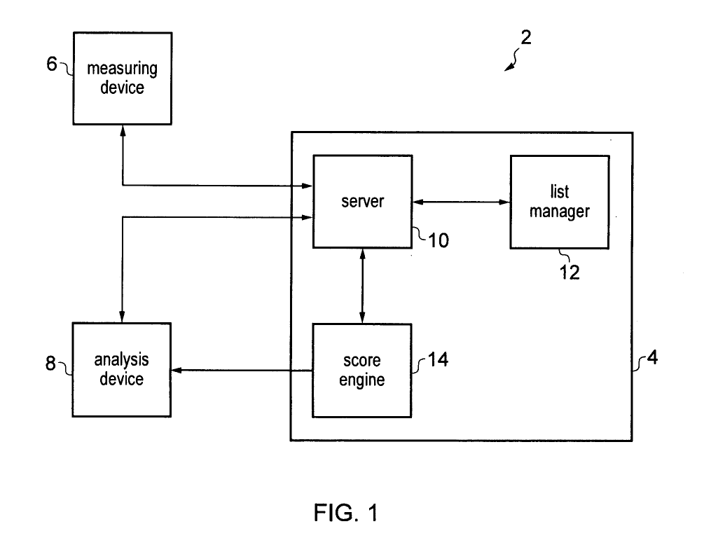

FIG. 1 illustrates a system 2 for monitoring the health of hospital patients and sending messages to medical practitioners when the health of a patient deteriorates. The system 2 comprises a base station 4, a measuring device 6, and an analysis device 8. In the present example embodiment, the base station 4 comprises a single personal computer or a network of interconnected personal computers which may be distributed around a hospital building or other location. The measuring device 6 and the analysis device 8 are each remote handheld mobile computing devices which are configured to communicate wirelessly with the base station 4, for example, via WiFi. It is to be understood that in some other example embodiments, the measuring device 6 and the analysis device 8 are configured to communicate with the base station 4 using alternative communication means or protocols.

The base station 4 comprises a server 10, a list manager 12 and a score engine 14. The server 10 is configured to communicate with both the measuring device 6 and the analysis device 8. The list manager 12 is configured to communicate with the server 10. The score engine 14 is configured to communicate with both the server 10 and the analysis device 8. The base station may be provided with an interface (not shown) so that a human user can communicate directly with the base station 4. For example, the interface may comprise a display screen and appropriate input devices, such as, a mouse and a keyboard.

Although FIG. 1 only shows a single base station 4, measuring device 6 and analysis device 8, it is to be understood that some example embodiments are provided with a plurality of one or each element. For example, in some example embodiments each doctor responsible for at least one patient is provided with their own analysis device 8. Additionally or alternatively, each nurse who is responsible for recording patient vital signs is provided with their own measuring device.

### Overview of Mobile Devices

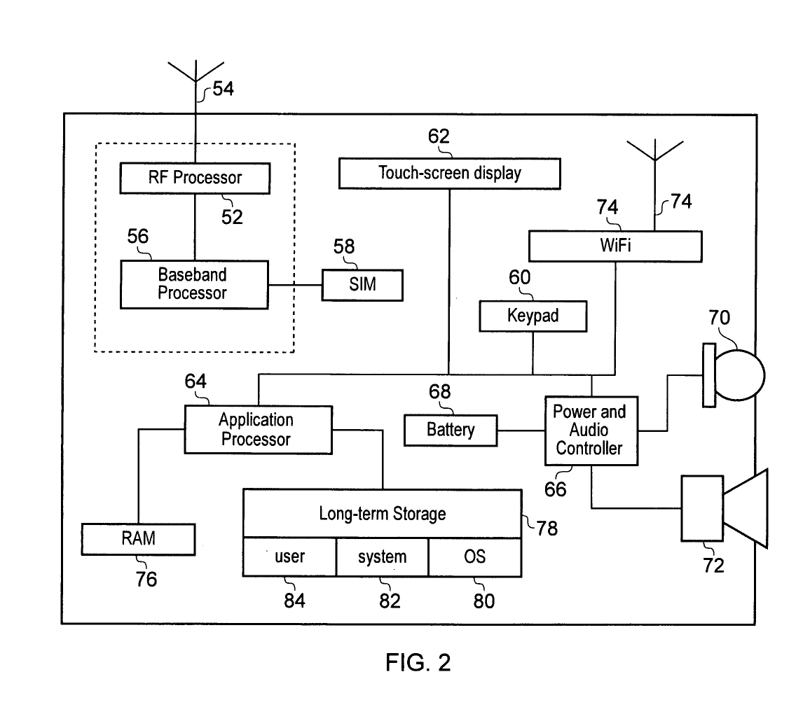

In the present example embodiment, the measuring device 6 and the analysis device 8 both comprise mobile computing devices. FIG. 2 shows a schematic view of some of the internal hardware elements of an exemplary mobile computing device. The device 2 comprises hardware to perform telephony functions, together with an application processor and corresponding support hardware to enable the device to have other functions, such as messaging, internet browsing, email functions, satellite navigation and the like. In FIG. 2, the telephony hardware is represented by the RF processor 52 which provides an RF signal to an antenna 54 for the transmission of telephony signals, and the receipt therefrom. Additionally provided is baseband processor 56, which provides signals to and receives signals from the RF Processor 52. The baseband processor 56 also interacts with a subscriber identity module 58, as is well known in the art. The telephony subsystem of the device 2 is beyond the scope of the present invention.

A keypad 60 and a touch-screen 62 are controlled by an application processor 64. A power and audio controller 66 is provided to supply power from a battery 68 to the telephony subsystem, the application processor 64, and the other hardware. The power and audio controller 66 also controls input from a microphone 70, and audio output via a speaker 72. Also provided is a WiFi antenna and associated receiver element 74 which is controlled by the application processor 64 and is capable of connecting to and communicating with a wireless computer, or computer network, in the vicinity of the device 2, such as, the base station 4.

In order for the application processor 64 to operate, various different types of memory are provided. Firstly, the device 2 includes Random Access Memory (RAM) 76 connected to the application processor 64 into which data and program code can be written and read from at will. Code placed anywhere in RAM 76 can be executed by the application processor 64 from the RAM 76. RAM 76 represents a volatile memory of the device 2.

Secondly, the device 2 is provided with a long-term storage 78 connected to the application processor 64. The long-term storage 78 comprises three partitions, an operating system (OS) partition 80, a system partition 82 and a user partition 84. The long-term storage 78 represents a non-volatile memory of the device 2.

In the present example, the OS partition 80 contains the firmware of the computing device which includes an operating system. Other computer programs may also be stored on the long-term storage 78, such as application programs, and the like. In particular, application programs which are mandatory to the device, such as, communications applications and the like, are typically stored in the system partition 82. The application programs stored on the system partition 82 would typically be those which are bundled with the device by the device manufacturer when the device is first sold. Application programs which are added to the device by a user would usually be stored in the user partition 84.

As stated, the representation of FIG. 2 is schematic. In Practise, the various functional components illustrated may be substituted into one and the same component. For example, the long-term storage 78 may comprise NAND flash, NOR flash, a hard disk drive or a combination of these. Additionally, some functional components illustrated may be absent. For example, the telephony subsystem may be omitted such that the mobile communication device is capable of communicating with external devices via WiFi.

### Overview of System Operation

Next will be described an overview of the operation of the system 2 of FIG. 1. The measuring device 6 is used by a hospital employee, such as a nurse, to periodically record a set of vital signs for one or more patients. Accordingly, the measuring device is a device for documenting data, such as, vital signs. The measuring device 6 wirelessly transmits to the server 10 of the base station 4 each set of vital signs input to the measuring device 6. The server 10 stores each set of vital signs received from the measuring device 6 so that data relating to an individual patient is retrievable therefrom. Additionally, the server 10 sends each received set of vital signs to the score engine 14. It is noted that each set of vital signs relate to the status of an individual patient at a single period in time. On receipt of a set of vital signs, the score engine 14 calculates a score or indicator to determine if the health of the patient corresponding to the set is deteriorating, improving or unchanged at the time period corresponding to the set. Where applicable, the score or indicator also indicates the extent of deterioration or improvement.

Depending on the calculated score or indicator, the score engine 14 sends a message to one or more medical practitioners in respect of the patient. The message may be one of a range of different message types, wherein each different message type indicates a different severity of deteriorating health. For example, if the health of the patient is deteriorating rapidly, a ‘critical’ alert may be sent to both a nurse associated with the patient and a doctor associated with the patient. Alternatively, if the health of the patient is deteriorating slowly, a ‘high’ alert may be sent only to the nurse associated with the patient. Alternatively, if the health of the patient is unchanged, no alerts may be sent to a medical practitioner in respect of the patient.

In order that the nurse and doctor associated with the patient can receive alerts relating to patients with whom they are associated, each has their own designated analysis device 8. The score engine 14 is capable of wirelessly communicating with each analysis device 8 so that a message can be sent thereto. Further, the score engine 14 is able to identify which nurses and doctors are associated with, or responsible for, each patient by consulting the list manager 12.

The list manager 12 is configured to determine which medical practitioner should be sent an alert when a particular patient's health deteriorates. Therefore, the list manager 12 contains information about which medical practitioners are associated with, or responsible for, which patients at a particular time. Specifically, each medical practitioner associated with, or responsible for, one or more patients is assigned a list comprising those one or more patients. It is the role of the list manager 12 to manage those lists and ensure that they are up-to-date. Furthermore, since medical practitioners will only be on duty some of the time, the list manager 12 is capable of determining which medical practitioner is responsible for each patient at any specific time. For example, a different doctor may be responsible for a particular patient during the day-shift, compared to the evening-shift or night-shift. Accordingly, once the score engine 14 identifies that a message needs to be sent to at least one medical practitioner in respect of a patient, the score engine 14 consults the list manager 12 to identify which medical practitioners are responsible for the patient at that time, and therefore, which medical practitioners must be sent a message.

According to the above-described operation, the system 2 provides a centralized server 10 which stores the medical records for each patient, such that up-to-date records for each patient are retrievable. The system 2 comprises a measuring device 6 through which the medical records of each patient may be periodically updated. The system 2 contains a score engine 14 which is capable of interpreting the input medical records for each patient, automatically identifying when the health of a patient is deteriorating, and determining the extend of the deterioration. The system 2 further comprises a list manager 12 which maintains a patient list for each medical practitioner, and knows when each medical practitioner is on duty. Therefore, the list manager 12 manages associations between medical practitioners and patients. Accordingly, if the score engine 14 identifies that a patient's health is deteriorating, it can send an alert message to the medical practitioners responsible for that patient at that time. Thus, the score engine sends messages to associated medical practitioners in dependence on the status of the patient's health. Those medical practitioners can receive their messages using an analysis device 8, consult the patient's records using the analysis device 8, and decide how to react. For example, the medical practitioner may choose to rush to the patient in order to administer emergency treatment. Alternatively, the medical practitioner may instruct a specialist to rush to the patient to administer specialist treatment. Alternatively, the medical practitioner may decide that the patient does not need emergency treatment at this time.

As mentioned above, the base station 4 may be provided with an interface so that a human user can communicate directly with the base-station 4. Specifically, the user may use the interface to review and manage patient data stored on the server 10, or list data stored on list manager 12, or score data stored on the score engine 14. For example, a medical practitioner may use the interface to set up a new list of patients under his supervision. Additionally or alternatively, the practitioner may amend an existing list to include or exclude particular patients. In some example embodiments, the base-station comprises a network of computers and the interface comprises an intranet running on that network, the intranet being operable by peripherals of at least one of the computers in the network.

It is to be understood that although FIG. 1 illustrates a single measuring device 6 and a single analysis device 8, any number of each element may be provided. For example, each medical practitioner responsible for recording vital signs may be provided with their own measuring device. Additionally, each medical practitioner responsible for at least one patient may be provided with their own analysis device. Additionally or alternatively, a hospital may own a pool of measuring devices and pool of analysis devices, so that any medical practitioner on duty can use one of the pool. Furthermore, a hybrid handheld device may be provided that can act as both a measuring device and an analysis device.

An advantage of the above-described system is that a patient's records can be constantly updated on a centralized server so that multiple people can have access to those records at different locations inside the hospital and outside the hospital. Another advantage is that a score engine is provided to automatically calculate a score or indicator to identify deteriorating health, each time a set of vital signs is input to the server. Therefore, the possibility of introducing human errors into the step of calculating the score or indicator is reduced. Another advantage is that a list manager is provided to maintain a patient list for each medical practitioner, and keep a record of when each medical practitioner is on duty. Accordingly, for any patient and at any time, the system can identify which medical practitioners are responsible for a patient and which medical practitioners should therefore be messaged. Another advantage is that each medical practitioner responsible for recording patient vital signs is provided with a measuring device, which is a handheld device that can be used at a patient's location, such as, their bedside, a corridor, or a waiting room. Accordingly, vital signs for a patient can be recorded quickly and accurately because they can be input onto the server in one action at the patient's location, i.e. they are not recorded on paper first and then input onto a computer at a later time. A further advantage is that each medical practitioner responsible for analysing a patient's records and administering treatment is provided with an analysis device, which is a handheld device that can be used at various locations inside and outside the hospital. Accordingly, the medical practitioner can be alerted that the health of one of their patient's is deteriorating at anyone of those various locations. Also, the medical practitioner does not have to be at the patient's location in order to review the patient's records and identify an appropriate course of treatment. Additionally, multiple medical practitioners can review a single patient's records from multiple different locations. In this way, it is possible for medical practitioners to focus their efforts on patients who require it most, or in other words, the medical practitioner's time is not wasted on patient's who do not need urgent attention when other patient's do require urgent attention.

### Measuring Device Operation

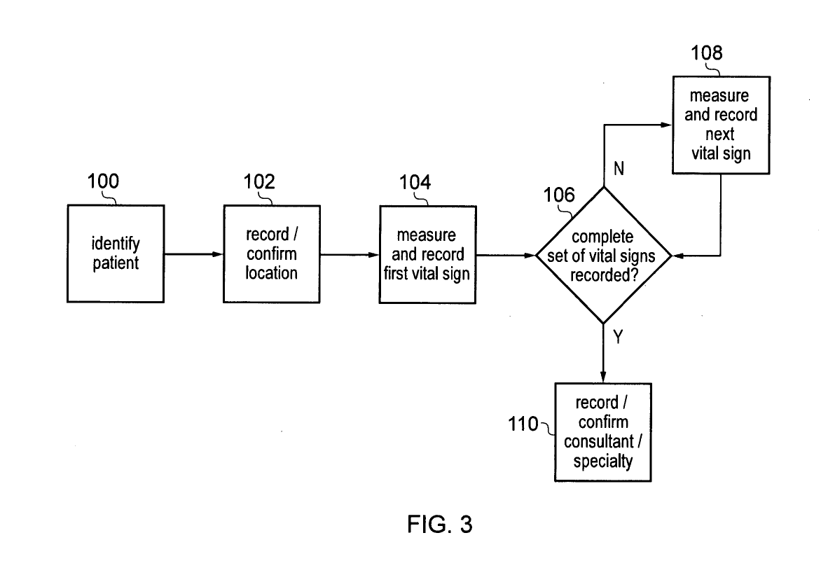

The following provides a more detailed explanation of the system 2, with reference to FIGS. 3 to 5.

FIG. 3 is a flow diagram illustrating the operation of the measuring device 6 with respect to recording the vital signs of a patient. In the present example embodiment, the process of FIG. 3 is performed by a medical practitioner, such as a nurse, using the measuring device 6.

When the nurse first starts using the measuring device, the nurse must log into the measuring device in a manner which will be apparent to the skilled person. For example, the nurse may use the keyboard 60 or touch-screen display 62 of the measuring device to input login details. Once the nurse has logged into the measuring device, the measuring device displays on the touch-screen 62 a list of patients for which the nurse is responsible for taking vital sign measurements. The measuring device also indicates additional information for each listed patient, including a score indicating whether or not the patients health is deteriorating. Additionally, the measuring device highlights any patients for which vital sign recordings are overdue. If vital sign recordings are not overdue for any patient the measuring device also specifies the time until the next vital sign recording session is due. Accordingly, the nurse can use the measuring device to identify which patients they must record vital signs for, and when those vital signs must be recorded. The measuring device is able to identify which patient's each nurse is responsible for by consulting the list manager 12. The measuring device is able to identify patient record information by communicating with the server 10.

Processing begins at step 100, wherein the logged-in nurse identifies the patient using the measuring device. For example, the patient may be assigned a barcode or an RFID tag on admission to the hospital and, at step 100, the nurse enters details of the barcode or RFID tag into the measuring device 6. For example, the measuring device 6 may include means for electronically reading the barcode or RFID tag. Alternatively, the nurse may simply enter the patient's name, or some other unique patient identifier, into the measuring device 6 using the keyboard 60 or touch-screen 62. Once a patient identifier has been entered, the measuring device 6 communicates with the server 10 of base station 4 to confirm that a patient matching the input identifier is a patient of the hospital. If the identifier does not match any patient records stored on the server 10, the measuring device 6 prompts the nurse to enter a different patient identifier. If the identifier does match patient records stored on the server 10, processing flows to step 102.

At step 102, the measuring device 6 instructs the nurse to record, or confirm, the location of the patient. If no location, or an incorrect location, is stored on the server 10 for the identified patient, the nurse will have to input an up-to-date location. For example, this could be the case if this is the first time vital signs have been recorded for the patient, or if the patient has just moved location. Alternatively, if vital signs have been recorded for the patient before and the patient has not moved then the location retrieved from the server 10 by the measuring device 6 will be accurate and therefore, the nurse must just confirm the stored location. Once the patient's location has been recorded or confirmed processing flows to step 104.

At step 104, the measuring device instructs the nurse to measure and record the first vital sign of the patient. In the present example embodiment, the measuring device instructs the nurse to record a set of different vital signs. Specifically, the measuring device instructs the nurse to record the following vital signs: pulse; temperature; systolic blood pressure; respiratory rate; neurological indicator (A.V.P.U.—best response on the following sliding scale: patient Alert, responds to Voice, responds to Pain, and patient Unresponsive); oxygen saturation; and, whether or not an oxygen mask has been administered (including which mask is used). Therefore, in the present example embodiment, at step 104, the nurse records the patient's pulse using the measuring device 6, following which processing flows to step 106. It is noted that a purpose of recording the set of vital signs is so that the score engine 14 can calculate a score or indicator which identifies if the patient's health is deteriorating, and the extend of any deterioration. In the present example embodiment, the algorithm for calculating the score or indicator uses the above-described vital signs to form the score. A detailed explanation of the algorithm is provided later.

At step 106, the measuring device 6 identifies whether or not the nurse has entered a complete set of vital signs. In the instant case, only one of the seven vital signs has been recorded. Therefore, processing flows to step 108. At step 108, the measuring device instructs the nurse to measure the next vital sign in the set, which in this case is the patient's temperature. Once the second vital sign has been measured, processing flows back to step 106, wherein the measuring device again identifies if a complete set of vital signs has been entered. Now, two of seven vital signs have been entered. Processing continues to flow in a loop around steps 108 and 106 until step 106 is reached when a complete set of vital signs has been recorded via the measuring device, at which point processing flows from step 106 to step 110.

At step 110, the measuring device instructs the nurse to record or confirm the patient's consultant and/or specialty. If this is the first time vital signs are recorded for the patient, or if the consultant or specialty has recently changed, the nurse is instructed to record the patient's consultant or specialty. On the other hand, if the patient's vital signs have been recorded before and the consultant and specialty have not changed, the measuring device instructs the nurse to confirm the patient's consultant or specialty. Following the operation of step 110, the measuring device 106 transmits the vital signs recorded by the nurse during the process flow of FIG. 3 to the server 10 of the base station 4. Alternatively, the information may be transmitted in one or more portions during the course of the above-described processing of FIG. 3. Additionally, if the nurse updates the consultant or specialty information then the server 10 sends the previous and new consultant and/or specialty a message to notify them of this update. Specifically, the server 10 sends a message to the analysis device of the previous and new consultant or specialty. The receipt and management of messages using the analysis device is described later.

Additional clinical data which is not used to calculate the score or indicator relating to patient health deterioration can also be captured as part of a ‘standard’ observation set of vital signs. The method of data capture on the measuring device is analogous to the above-described method. Further, such additional data may vary between hospitals, or between areas within the same hospital. For example, a pain score may be captured throughout a hospital as part of the standard observation set, however, specialised observations such as cardiac rhythms may only be configured as a standard observation on an appropriate ward.

An advantage of the above-described method of data capture is that it is faster and more accurate than pen and paper recordal. For example, errors are not introduced by poor handwriting or lost paper. Further, the above system enables patient location to be maintained more accurately than conventional systems. Specifically, conventional systems rely on an administrative system, such as a hospital patient administration system (PAS), to keep an up-to-date record of the location of each patient. This conventional system is predominantly used for clerical operations, such as invoicing, and therefore, it is frequently not updated as a matter of urgency. Furthermore, it is usual that the conventional system is maintained by clerical staff who only work during normal office hours, for example 09:00 to 17:00, Monday to Friday, and therefore, records of patient's location outside these hours can be inaccurate.

In some example embodiments, the measuring device is configured to only submit a set of vital signs if a measurement for each vital sign in the set is provided. In this way, the measuring device prevents incomplete sets of vital signs being submitted. Accordingly, the score engine only calculates score values which are as accurate as possible. In other words, the score indicator does not calculate a score value using only a subset of the information required. In some other example embodiments, the measuring device may submit an incomplete set of vital signs. For example, submittal of an incomplete set of vital signs may be permitted if reasons for non-completion are documented. In such cases, default or nil values may be used for any unmeasured vital signs. Additionally, a score calculated using an incomplete set of vital signs may indicate that it is an incomplete score.

### Analysis Device Operation

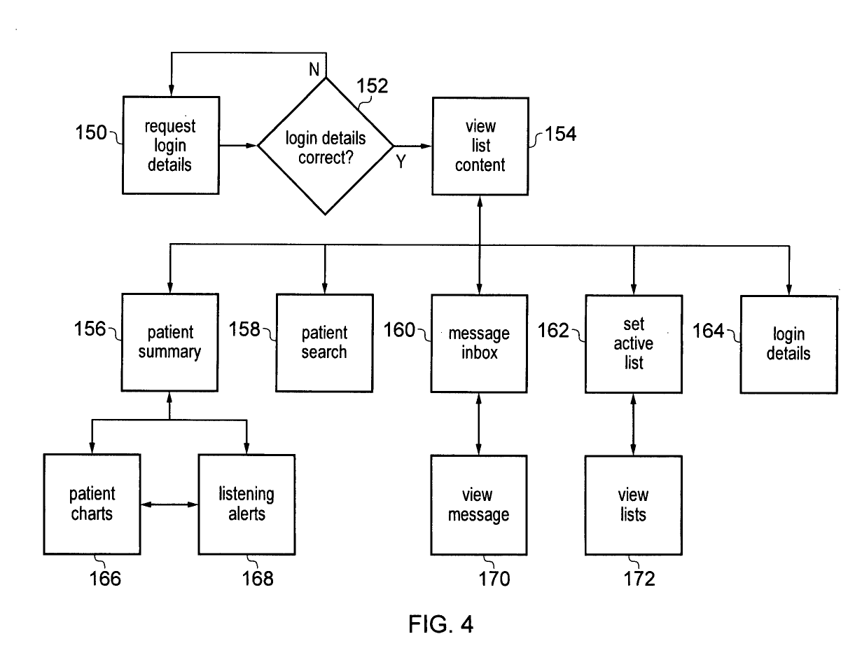

FIG. 4 is a flow diagram illustrating the operation of the analysis device 8. In the present example embodiment, the analysis device 8 is operated by a hospital employee, such as a doctor in charge of a group of patients. The following describes aspects of the internal operation of the analysis device and a user-interface displayed on the screen of the analysis device.

Processing begins at step 150, wherein the doctor begins using the analysis device 8 after a period of non-use. Upon activating the analysis device 8, the analysis device instructs the doctor to enter login details. At step 152, the analysis device checks that the login details entered by the doctor are valid. If the login details match recognised details stored on the server 10 of base station 4, the login process is complete and processing flows to step 154, otherwise processing flows back to step 150. It is to be understood that once logged-in the doctor can logout of the analysis device by selecting an appropriate logout button, for example, via the keyboard 60 or touch-screen display 62.

At step 154, the analysis device 8 displays on the touch-screen 62 the contents of a list of Patients assigned to the logged-in doctor. The default list of patients displayed by the analysis device is the doctor's active list. The analysis device uses the login details provided at step 150 to identify which doctor has logged in. The analysis device then consults the list manager 12 in order to identify the active list of patients for the identified doctor. The doctor's active list comprises a list of all the patients which the doctor is responsible for during his current shift, i.e. the active list is the doctor's on-duty list. Once the active list is displayed on the touch-screen of the analysis device, the doctor may choose to arrange the list according to different attributes of the patients in the list by using appropriate buttons on the keyboard or touch-screen. For example, buttons may be provided to order the list by patient name, patient location, the number of outstanding actions per patient, or a patient score indicating whether the patient's health is deteriorating.

Each entry in the active list relates to a different patient. Also, each entry in the active list provides additional patient information relating to the corresponding patient. For example, the additional patient information displayed may include, a listening flag, a score indicating the patient's health, a patient location, a name of a consultant responsible for the patient. Furthermore, the score may be accompanied by an up arrow or a down arrow indicating whether the patient's health is getting better or worse since the last observations were taken, i.e. since the last vital signs were recorded. The listening flag will be discussed in greater detail below in connection with listening alerts.

It is to be understood, that the analysis device can use the view list content screen to display the contents of any patient list visible to the doctor who is logged-in to the analysis device. In addition to viewing groups of patients by patient lists, the doctor can also view groups of patients by their location. For example, the doctor can use the view list content screen to view the group of patients on a particular hospital ward. The process of viewing the contents of a list other than the active list will be described later.

From the view list content screen (step 154), the doctor can navigate to a variety of different screens (steps 156 to 164). Specifically, the doctor may instruct the analysis device to navigate to the patient summary screen (step 156), the patient search screen (158), the message inbox (step 160), the set active list screen (step 162), or the login details screen (step 164). In the present example embodiment, the doctor can navigate to these other screens by selecting an appropriate soft-key displayed on the touch-screen of the analysis device. In other example embodiments, different navigation methods may be provided by the analysis device, such as, for example, voice control and hard-keys on the keyboard. The following describes each different screen displayed by the analysis device on its touch-screen.

It is to be understood that each screen displayed by the analysis device touch-screen includes a menu. In the present example embodiment, the menu is located at the bottom of the screen, however, in some other example embodiments the menu may be located elsewhere on the touch-screen. The menu includes a plurality of soft-keys which can be selected by the doctor to cause the analysis device to navigate to specific screens. In the present example embodiment, there is a soft-key for navigating to each of the following screens: the view list content screen (step 154), the message inbox screen (step 160), the set active list screen (step 162), the patient search screen (step 158), and the login details screen (step 164).

### Patient Summary Screen

The doctor can cause the analysis device touch-screen to display the patient summary screen (step 156), by selecting a patient in the active list. Selection is achieved by pressing on a portion of the touch-screen which displays information relating to the patient. At step 156, the patient summary screen relating to the patient selected by the doctor is displayed on the analysis device touch-screen. In general, the patient summary screen provides a snap-shot of the patient's condition. Specifically, the patient summary screen is a list split up into a number of different list portions.

Additionally, the analysis device displays above all the list portions a soft-key for adding or removing the patient from one or more of the doctor's patient lists. As will be described in greater detail below, the doctor has one or more patient lists. Each list comprises a group of patients which the doctor can be responsible for. At any time, one or more of the doctor's lists can be designated ‘active’, meaning that the doctor is responsible for those patients until the ‘active’ status is removed. Accordingly, the doctor's active list or lists are his on-duty list or lists. While a list is active the doctor will be send messages in respect of each patient in the list, periodically keeping him updated with the patient's status. Activating the soft-key causes the analysis device to display a further page which lists all of the doctor's lists and highlights any which include the patient. The doctor can then add or remove the patient from each of his lists. In the present example embodiment, the active list or lists are displayed above all other lists to make them more prominent. The analysis device displays a further soft-key which enables the doctor to cause the analysis device to navigate back to the patient summary screen.

It is noted that the analysis device 8 consults the list manager 12 to identify a doctor's list or lists. Further, when modifications are made to lists on the analysis device 8, these modifications are transferred from the analysis device 8 to the list manager 12 such that the list manager is always up-to-date. In some example embodiments, depending on the patient or the doctor, the list manager 12 only allows the patient to be part of a predetermined number of doctors' active lists. For example, the list manager may only permit certain patients being part of one doctor's active list. In this case, when a new doctor adds the patient to their active list, and the patient is already part of another doctor's active list, the patient is automatically removed from the other doctor's active list. In this case, the other doctor is sent a message to his message inbox (described later) informing him that he is no longer responsible for the patient. In a further example embodiment, however, the list manager may permit one or more patients to be part of any number of doctor's active lists. In such examples, therefore, when a doctor adds one of those patients to their active list, and that patient is already part of another doctor's active list, the patient is not automatically removed from the other doctor's active list.

The following now describes the above-mentioned list portions displayed as part of the patient summary screen. A first list portion displays identification data, such as, for example, the patient's full name, the patient's medical number, the patient's hospital number, the patient's date of birth, the patient's height and weight, and the patient's gender.

A second list portion displays data relating to the patient's current stay in the hospital, such as, for example, the patient's location in the hospital, a ward-phone extension number, the consultant and/or specialty assigned to the patient, the patient's length of stay, the patient's admission date, and when the patient's next observation is due.

A third list portion displays data relating to notifications about the patient. Said notifications may include: listening alerts, a score indicating whether the patient's health is deteriorating, and a pain score. As will be described in further detail below, a doctor may use the analysis device to set up a listening alert for a particular patient to cause the server 10 to transmit a message to the doctor's analysis device when a particular characteristic of the patient changes, for example, by going above one threshold or below another threshold. The score indicating whether the patient's health is deteriorating will also be described below in greater detail. The pain score comprises an indication of how much pain the patient is in, such as, for example, a numerical value between 0 and 10, where 0 indicates no pain and 10 indicates maximum pain.

A fourth list portion displays data relating to patient alerts, patient actions, and patient information. Each different category (i.e. alert, action or information) is separately identifiable, for example, by a different shaped or coloured icon. For example, alerts are identified by a triangular icon which is red if the alert is critically important, and amber otherwise. In the present example embodiment, alerts notify the doctor of very important information relating to the patient. For example, red alerts include a notification that the patient has contracted MRSA, or that the patient has an allergy to a particular medication. Amber alerts may include a VIP score level indicating the status of a cannula inserted in the patient's body. Actions may be identified by a circular icon. Actions provide the doctor with a reminder, such as, for example, an action may be to perform a VTE assessment, or to remove or examine a cannula inserted in the patient's body. Information may be identified by a square icon. Information gives the doctor useful information about the patient, such as, for example, the location of a cannula inserted into the patient, the date and time when the cannula was last checked, or information on the patient's medical condition, like whether it is a long term illness.

A fifth list portion displays data relating to special observations. As mentioned previously, special observations may be recorded in addition to the set of vital signs used to calculate the score indicating if the patient's health is deteriorating. Special observations may relate to the following: analgesia, bowels, CVP, diabetic monitoring, epidural, neurological, pre and post nebuliser, sedation, urine, and wounds and drains. These observations are designated special because they are not directly included in the calculation of the score indicating health deterioration. Special observations may be important because of the particular patients physiology or because of a particular course of medical treatment. Each special observation may be recorded with a date and time when they were measured. In some example embodiments, only the last five special observations are recorded. Further, in some example embodiments, special observations are only shown while they recent, for example, special observations older than 14 days may not be show.

A sixth list portion displays pathology data relating to the patient. In the present example embodiment, the pathology data is organised by data type and the date it was collected. The pathology data presented may be limited to the most recent data collected. For example, only pathology data which was collected less than a certain time period ago, such as two weeks, may be presented. Alternatively, only the most recent five pathology reports may be shown. Exemplary pathology data includes: liver function test results, full blood count results, or toxin test results. The doctor can select each of the pathology entries listed to cause the analysis device to display on the touch-screen the actual test results for that entry. The displayed page specifies the date the test was administered, i.e. the date a sample was taken. Additionally, the displayed page highlights any results which are outside a normal range to assist the doctor in making an accurate diagnosis and prescribing an appropriate treatment.

In the present example embodiment, the information in the list is arranged in order of importance. For example, the whole list may be in order of descending or ascending importance. Alternatively, each different list portion may be displayed in the above-described order, or any other order, and within each list portion the data may be displayed in order of descending or ascending importance. In some other embodiments of the invention, the icons used to identify between alerts, actions and information may be different from those described above. Further, in some other example embodiments, other list portions may be provided for displaying other medical data, such as, for example, radiology results. Also, in some other example embodiments, one or more of the above-described list portions may not be displayed. For example, if there are not pathology results for a particular patient, the list portion related to pathology reports may be omitted from the patient summary.

In addition to the above, the patient summary page displays soft-keys to cause the analysis device to display a patient chart screen (step 166), and a listening alerts screen (step 168). The doctor is able to operate the analysis device 8 to cause processing to flow from step 156 to step 166 by selecting the patient chart soft-key from the patient summary screen.

A plurality of different charts are displayed on the patient chart screen. Each different chart may be selected for display on the touch-screen of the analysis device 8 by selecting a different one of a plurality of soft-keys provided on the patient chart screen. In the instant case, four soft-keys are provided, however, in some other embodiments a different number of soft-keys are provided. One of the charts is set to be the default chart which is automatically selected for display when the patient chart screen is activated. Thereafter, the chart displayed corresponds to the soft-key which has been selected on the patient chart screen. In the present example embodiment, the following patient vital signs may be selected for display in graphical form: temperature, blood pressure, pulse, respiratory, AVPU (Neurological status), O₂ saturation level, mask type, and flow rate. Each of these vital signs is plotted against time and date, i.e. each vital sign value is plotted against the time and date when it was recorded. It is to be understood that in some other example embodiments, patient vital signs may be plotted against something other than date and time. Furthermore, in some other example embodiments, different vital signs may be plotted. Since in the instant case only four soft-keys are displayed on the touch-screen, multiple vital signs are plotted on the same graph. In some other embodiments, each vital sign may be displayed on a separate graph to all other vital signs.

In the present example embodiment, one of the plurality of soft-keys provided on the patient chart screen causes the analysis device to display a numerical chart on the touch-screen. In particular, the table comprises a row for each different patient vital sign and a column for each different date and time when the patients' vital signs were recorded. Accordingly, the table displays the recorded vital signs of the patient which were taken by the nurse using the measuring device 6, as described above with the reference to FIG. 2. Since the nurse records vital signs in a set, each column represents the recorded values of a set.

Since a great many measurements may be taken for each vital sign in respect of one patient, the analysis device is configured to display only a certain amount of historical data. For example, the analysis device may be configured to display only the last fourteen days worth of recorded values. Alternatively, the analysis device may be configured to display only a certain number of the most recent recorded values, for example, the five most recent values.

As mentioned above, the patient summary page displays soft-keys to a patient chart screen (step 166), and a listening alerts screen (step 168). The soft-key to the patient chart screen has been described above. The doctor is able to cause the analysis device to navigate to the listening alert screen from either the patient summary screen or the patient chart screen. Specifically, the doctor is able to operate the analysis device 8 to cause processing to flow from step 156 or step 166 to step 168 by selecting the listening alert soft-key.

The listening alert screen provides the doctor with the ability to set a listening alert for the patient so that if any specified physiological changes occur in respect of the patient, the server 10 sends a message to the doctor's analysis device. In the present example embodiment, the analysis device permits a doctor to see and review only listening alerts which he has created. In some other embodiments, the analysis device permits the doctor seeing listening alerts of one or more other doctors. The listening alert screen displayed on the touch-screen comprises a soft-key for creating a new listening alert. The doctor can select the soft-key to create a listening alert. Specifically, when the doctor activates the soft-key, the analysis device presents one or more vital signs to choose from. In the present example embodiment, the following vital signs can be chosen from: temperature, pulse, systolic blood pressure, diastolic blood pressure, respiratory rate, GCS and O₂ saturation. In some other embodiments, the number and type of possible vital signs can be different to those of the present example embodiment.

If the doctor selects one of the available vital signs, the analysis device prompts him to input a value of a first threshold and a second threshold. When the selected vital sign falls below the first threshold, the listening alert triggers, causing a message to be sent to the doctor's analysis device. Conversely, when the selected vital sign rises above the second threshold, the listening alert triggers, causing a message to be sent to the doctor's analysis device. In the present example embodiment, the doctor can choose to set either or both of the first and second thresholds. In some other embodiments, it is mandatory that the doctor sets the first and the second thresholds. In some other embodiments, more or less than two thresholds may be set. It is to be understood that when the doctors creates a new listening alert on the analysis device, the analysis device transmits details of the listening alert to the server 10 of base station 4. Since the server maintains an up-to-date record of the patients vital signs, the server becomes aware when a listening alert threshold it passed. At that time, the server sends the analysis device an appropriate message.

Once the doctor has set either or both of the thresholds, the analysis device navigates back to the listening alert screen. In addition to the ‘create new listening alert’ soft-key, the listening alert screen also provides a screen portion for displaying listening alerts which have been created. Obviously, if no listening alerts have been setup then none are shown on the listening alert screen portion. If listening alerts have been set up then they are displayed on the touch-screen as follows.

A first list portion contains an entry for each listening alert which has triggered. Each of these entries shows corresponding threshold values, and the date and time when the listening alert was triggered, along with the value that caused the trigger. A second list portion contains an entry for each listening alert which has been set but not triggered. Each of these entries shows corresponding threshold values. Those listening alerts listed in the first list portion may be selected by the doctor for removal by touching the portion of the touch-screen in which they are displayed. Those listening alerts listed in the second portion may be selected by the doctor for editing or removal in the same way. In the present example embodiment, the first list portion is listed before the second list portion so that triggered listening alerts are more prominent. In some other example embodiments, the listening alerts may be displayed and arranged differently.

### Patient Search Screen

The doctor can choose cause the analysis device touch-screen to display the patient search screen (step 158) from, for example the view list content screen 154, by selecting the appropriate soft-key on the menu. The patient search screen instructs the doctor to enter a patient number or a hospital number in order to search for a particular patient. If the entered number matches details of a patient stored on the server 10, the patient search screen presents the name, date of birth, patient number and hospital number of the stored patient. Further, the analysis device also displays a soft-key which, if pressed by the doctor, causes the analysis device to navigate to the patient summary page for the identified patient. Additionally, a cancel soft-key is provided if the doctor does not want to navigate to the patient summary page. Selecting the cancel soft-key navigates back to the patient search screen. Obviously, if no details stored on the server 10 match the input details, the patient search screen informs the doctor that no patient having the input details is known.

In some other example embodiments, the patient search facility is capable of searching for patients using additional or alternative search criteria to a patient number or a hospital number. For example, in some example embodiments it is possible to search by patient surname or some other unique patient identifier.

### Message Inbox Screen

The doctor can cause the analysis device touch-screen to display the message inbox screen (step 160) from, for example, the view list content screen (step 154), by selecting the appropriate soft-key on the menu. The message inbox screen displays a list of all messages sent to the doctor who has logged-in to the analysis device. Messages which have not been read or acted upon are highlighted, for example, by a coloured dot; or an icon. The doctor can select one of the messages listed in order to navigate to a screen displaying the details of that message. In the present example embodiment, the following message details are displayed: the message sender; any other recipients of the message; the message title; details of the patient to which the message relates; and, message content. Further, soft-keys are displayed which when activated cause the following actions: navigate to a patient summary page for the patient to which the message relates; navigate back to the message inbox; navigate to the details of the next message listed in the message inbox; navigate to the details of the previous message listed in the message inbox; and delete the message.

In addition to the above, depending on the message type, the following operations are also supported. If the message contains test results, such as pathology results, a summary of these results is displayed in the body of the message, and a soft-key is provided to cause the analysis device to navigate to the complete pathology results. If the message relates to a triggered listening alert then the following information is displayed in the body of the message: the vital sign which triggered the listening alert; a soft-key to navigate to the listening alert screen; and, a soft-key to remove the listening alert. It is to be understood that messages may be sent to medical practitioners in respect of a patient in response to identified deterioration of the patient's health or for some other reason, such as, for example, because new test results are available for the patient.

In the present example embodiment, the message inbox screen only stores the most recent messages, such as, for example, the most recent two hundred messages. Additionally, messages over a certain age are automatically deleted, i.e. messages over, for example, seven days old, are automatically deleted. In some other example embodiments, a different number of messages may be stored and automatically deleted. Additionally, in some other example embodiments, different message details, or soft-keys, are displayed in the message inbox screen and the individual message screen.

In the present example embodiment, the analysis device is capable of notifying the doctor when a new message is received. For example, to indicate that a new message has been received the analysis device may play a sound, vibrate, or display a notification on the touch-screen.

In the present example embodiment, if the system identifies that a patient's health is deteriorating and therefore, that a message must be sent in this regard, only medical practitioners associated with the patient are sent a message. Specifically, only medical practitioners having an active patient list containing the patient are sent a message. Therefore, the analysis device receives messages that are sent to the medical practitioner logged-in to the analysis device, and are sent in respect of patients in that medical practitioner's active list.

### Set Active List Screen

The doctor can cause the analysis device touch-screen to display the set active list screen (step 162) from, for example, the view list content screen (step 154), by selecting the appropriate soft-key on the menu. The set active list screen comprises two list portions. A first list portion provides a soft-key which allows the doctor to specify that no lists are active. A second portion provides a scrollable list of all the available lists. Further, in the second list portion, each list which is designated as ‘active’ is highlighted in some way, such as, being in a different font or colour. The analysis device is able to consult the list manager 12 to identify whether or not active lists have been designated, and if so, what the list contents are. The analysis device also consults the list manager 12 to identify all lists which are visible to the doctor. In the present example embodiment, doctors can create patient lists, and in so doing, designate them as either ‘public’ or ‘private’. The second list portion displays all the patient lists which are public, as well as, private lists which have been created by the doctor currently logged-in to the analysis device. In some other example embodiments, lists may be displayed differently. For example, only public or private lists may be displayed. Alternatively, the second list portion may be split into two further portions, one including all visible public lists, and the other including all visible private lists.

Selecting one of the lists in the second portion of the set active list screen causes the analysis device to present an option to the doctor to set that list as an active list. The analysis device displays soft-keys so that the doctor can either confirm and view the selected list, or cancel the request. In the present example embodiment, multiple lists can be set active. Setting a list active means that messages relating to all patients in the list will be sent to the doctor's message inbox by the server. For example, listening alert messages, alerts that the score indicating deterioration of patient health has increased, and test result messages will be sent to the doctor's messaging inbox. In some example embodiments, only up to a predetermined number of lists may be set as active.

Selecting the soft-key in the first list portion enables the doctor to specify that no lists are set as ‘active’. If the doctor selects the ‘no active list’ soft-key then the analysis device displays soft-keys so that the doctor either confirms or cancels the request. In the present example embodiment, if the doctor confirms that no lists are to be set active then the analysis device displays a confirmation screen warning the doctor that they will not receive messages in respect of any patients.

The set active list screen further comprises a soft-key to the view list screen (step 172). The doctor can select the soft-key in order to cause processing of the analysis device to flow from step 162 to step 172. The view list screen comprises two list portions. A first list portion contains all the doctor's active lists. A second list portion contains all the other patient lists which are visible to the doctor currently logged into the analysis device. The doctor can select a patient list in either list portion to cause the analysis device to display the view list content screen (step 154) relating to the selected list. In other words, the analysis device navigates to a page showing the contents of the selected list. The arrangement of the view list content screen is described above.

### Login Details Screen

The doctor can cause the analysis device to display the login details screen (step 164) from, for example, the view list content screen (step 154), by selecting the appropriate soft-key on the menu. The login details screen provides the doctor with a option to change his login details, including his password. The login details screen first prompts the doctor to provide their existing password. Provided that the existing password is entered correctly, the analysis device prompts the doctor for a new password, and then prompts the doctor to confirm the new password. Assuming that the confirmation matches the new password, the new password is saved and the analysis device presents the doctor with a confirmation message. If the confirmation does not match the new password, an appropriate failure message is presented to the doctor and the password is not changed.

### Score Engine Operation

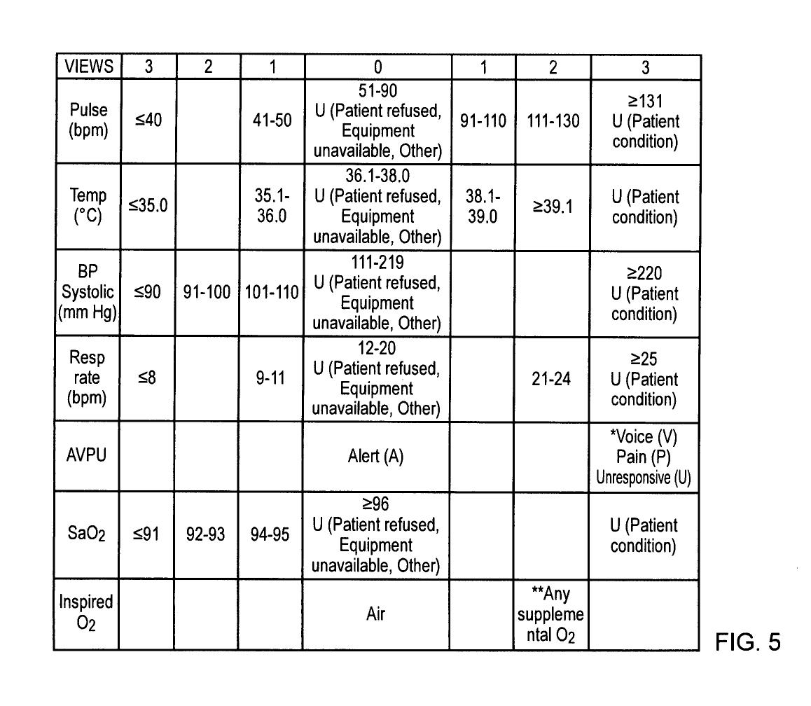

FIGS. 4 and 5 define the score for indicating deteriorating health of a patient. In the present example embodiment, the score is an early warning score (EWS), and in particular, VitalPAC Early Warning Score (VIEWS). FIG. 5 shows a table, wherein each row relates to a different observed vital sign. Specifically, the following vital signs are listed: pulse, temperature, blood pressure, respiration rate, the neurological indicator AVPU (patient is Alert, patient responds to Voice, patient responds to Pain, or patient is Unresponsive), oxygen saturation, and inspired oxygen. Each column relates to a particular score value between zero and three. In particular, each score column specifies the vital sign values necessary for the score corresponding to the column. For example, a pulse less than 40 bpm is designated a score of 3; a pulse between 41 bpm and 50 bpm is designated a score of 1; a pulse between 51 bpm and 90 bpm is designated a score of 0; a pulse between 91 bpm and 110 bpm is designated a score of 1; a pulse between 111 bpm and 130 bpm is designated a score of 2; and, a pulse greater than 131 bpm is designated a score of 3. Accordingly, an exemplary measured pulse of 60 bpm would be designated a score of 0.

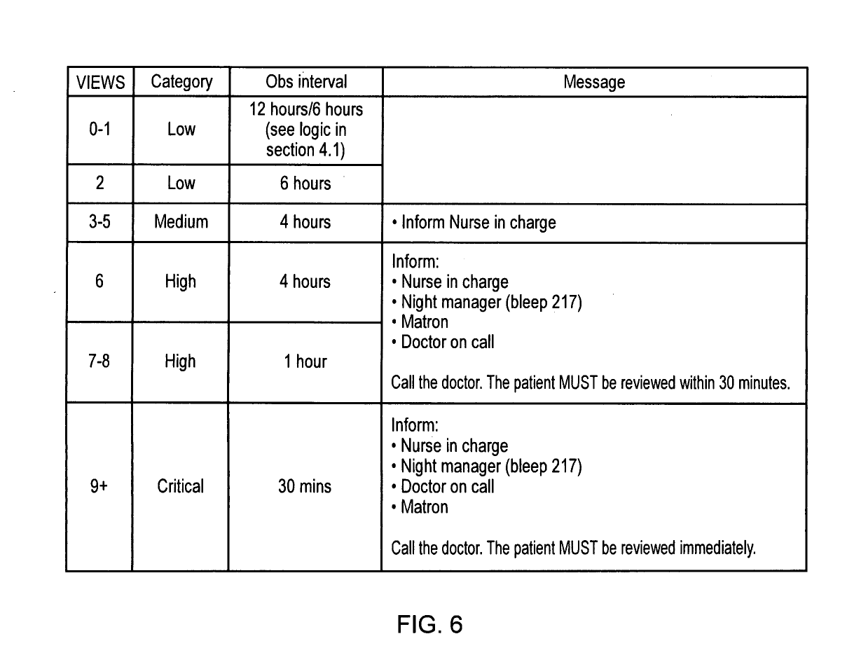

Once a set of vital signs have been input to the server 10 using a measuring device 6, as described above with reference to FIG. 3, the score engine 14 receives the vital sign measurements and calculates an overall EWS for the set. Specifically, the score engine first calculates a score for each vital sign in the set. Secondly, the score engine combines all the scores relating to the set to form an EWS. Depending on the value of the EWS, the engine either sends out messages in respect of the patient to which the vital signs relate, or does not. The score engine consults the list manager 12 to identify which hospital employees are responsible for which patients, i.e. the score engine consults the list manager to identify to whom the messages should be sent. The following describes this messaging operation with reference to FIG. 6.

Generally, in dependence on the value of the EWS, the score engine sends messages to a hospital employee or a particular group of hospital employees, and those messages indicate when the patient must be reviewed, and the regularity of further reviews thereafter. Accordingly, messages are sent in dependence on the status of the patient's health, such as, for example, in dependence on deteriorating health of the patient. Each message includes at least information for identifying the patient to which it relates and the EWS value. The following describes the actions which occur at various EWS threshold values.

In the present example embodiment, an EWS of nine or more indicates a ‘critical’ state. In this state, a message is sent to the following group of hospital employees: the nurse in charge, the night manager, the doctor on call, and the matron. The message to the doctor states that the patient must be seen immediately. Additionally, the patient list of the nurse responsible for recording the patient's vital signs is updated to instruct the nurse to record the patient's vital signs every thirty minutes.

An EWS of seven or eight indicates a ‘high’ state. In this state, a message is sent to the following group of hospital employees: the nurse in charge, the night manager, the doctor on call, and the matron. The message to the doctor states that the patient must be reviewed within thirty minutes. Additionally, the patient list of the nurse responsible for recording the patient's vital signs is updated to instruct the nurse to record the patient's vital signs every hour.

An EWS of six indicates a ‘high’ state. In this state, a message is sent to the following group of hospital employees: the nurse in charge, the night manager, the doctor on call, and the matron. The message to the doctor states that the patient must be reviewed within thirty minutes. Additionally, the patient list of the nurse responsible for recording the patient's vital signs is updated to instruct the nurse to record the patient's vital signs every four hours.

An EWS of three to five indicates a ‘medium’ state. In this state, a message is sent to the nurse in charge. Additionally, the patient list of the nurse responsible for recording the patient's vital signs is updated to instruct the nurse to record the patient's vital signs every four hours. An EWS of two indicates a ‘low’ state. In this state, no messages are sent. However, the patient list of the nurse responsible for recording the patient's vital signs is updated to instruct the nurse to record the patient's vital signs every six hours. An EWS of zero or one also indicates a ‘low’ state. As before, no messages are sent. However, the patient list of the nurse responsible for recording the patient's vital signs is updated to instruct the nurse to record the patient's vital signs every six to twelve hours.

According to the above, the score engine 14 is able to receive vital signs relating to a patient from the server, calculate an EWS value to identify the current state of the patient's health, and then send messages to relevant hospital employees to notify them of the deteriorating health of a patient under their supervision, and in some cases, instruct them to review the patient within a certain time limit.

In order that the response to an EWS message is coordinated, there are two phases to responding. In the first phase, the doctor or nurse who has received an EWS message in respect of one of their patients, and who has decided to go to the patient, must indicate that they are ‘on their way to the patient’ to any other person who has received a similar EWS message. In this way, doctor or nurse time is not wasted responding to an EWS message which has already been, or is being, dealt with by another person. In the second phase, the first person to respond to the EWS message, i.e. the person to perform the first phase, must indicate that he has seen the patient, with the details of any intervention, to any other person who received a similar EWS score. The following describes the mechanism for responding in detail.

As discussed above, a medical practitioner, such as a nurse, can use a measuring device to record a set of vital signs relating to a patient. The measuring device communicates the recorded set of vital signs to the score engine. On receipt of the set of vital signs the score engine calculates a score, the value of which indicates the status of the patient's health. In dependence on the value of the score, the score engine sends messages to a hospital employee or a particular group of hospital employees, and those messages indicate when the patient must be reviewed, and the regularity of further reviews thereafter. Additionally, the score engine sends a message to the nurse's measuring device to keep the nurse abreast of the situation. For example, if the score value indicates that the patient is in need of immediate medical attention such that one or more doctors are sent messages in respect of the patient, the message to the nurse's measuring device (i.e. the measuring device to which the nurse is logged-in) includes the following information: patient identification information, the score value calculated using the vital signs just recorded, a deadline by which time the patient needs to be reviewed, and the one or more doctors which have been sent messages.

Considering the messages sent to the one or more doctors, each doctor will receive a message provided that they are logged into an analysis device which is in communication range with the rest of the system, e.g. the base-station. As mentioned above, the analysis device may vibrate, or play an audible tune, to indicate that a new message has been received. Included in the message is information that identifies the patient, the score value which has prompted the message, the time by which the patient needs to be reviewed, and the other doctors which were sent the same message. Also included in the message are a number of buttons which the doctor can select in order to complete the first phase of responding. The buttons allow the doctor to send a message to the nurse and the other notified doctors indicating that he is going to take an action based on the message, i.e. he is taking personal responsibility for progressing the care of the patient. For example, the buttons may allow the doctor to send a message indicating that he is en route to the patient; he is going to call the patient's ward; he cannot review the patient; he has given advice over the phone regarding the patient; he has reviewed the patient; or the patient does not require a review. According to this operation the nurse, who having just recorded the patient's vital signs will likely be at the patient's side, is kept up-to-date about whether or not the patient is going to be reviewed, and if so, by whom.

As mentioned above, following vital sign recording, the nurse is sent a message from the score engine so that the nurse is kept abreast of the situation. A set time period after receipt of this message, the measuring device displays a notification to the nurse. If the one or more doctors have responded as described above, the notification highlights the responses received. If none of the doctors have responded then the notification highlights that this is the case. Additionally, if none of the doctors have responded, or if the responses indicate that the patient is not going to be reviewed, the notification instructs the nurse to notify a suitable medical practitioner using different means, such as, for example, a bleeper or telephone system. In these circumstances, a suitable medical practitioner may be identified as the head doctor on duty, or one or a number of designated persons who should be called in these specific circumstances.

In the event a doctor has indicated that they are going to call the patient's ward, or are going to review the patient themselves, once this is done, the doctor must send a message to the nurse and the other notified doctors that the patient has been reviewed. This message may include details of the treatment administered or instructed by the doctor. This message may be sent in the same fashion as described above, i.e. the doctor may select one of the buttons from the original warning message.

According to the above described operation, the or each doctor notified of the deteriorating health of one of their patients can quickly and simply notify other doctors responsible for the patient, and the nurse currently caring for the patient, that he is going to review the patient, or he is not going to review the patient. Furthermore, the nurse currently caring for the patient is provided with a specific time period during which he can wait for a response from a notified doctor. The time period may be about two minutes. The nurse is then notified once the time period has expired so that the nurse can then seek alternative ways of locating a doctor who can review the patient. An advantage of this operation is that notified doctors are given an opportunity to volunteer to review one or their patients, however, if they are unable to volunteer, or do not notice the request, the patient is not kept waiting indefinitely. Accordingly, the patient is provided with better care.

It is to be understood that the above method of responding is also applicable to other messages received by one or more doctors. For example, a group of doctors may receive a message relating to test results of a patient, such as, blood test results. Accordingly, one of the doctors could use the above-described method of responding to notify the other doctors in the group that he is going to take responsibility for progressing the care of the patient relating to the test results.

In some example embodiments, a different set of observations may be measured to those described above. For example, some of the above observations may be omitted, alternative observations may be included, or a combination of these two possibilities may be true. Also, the alternative observations may be other physiological measurements, or may be some other type of patient measurement. For example, Alternative observations may include: a blood-glucose measurement, a cardiac rhythms, a peak flow measurement, or a hydration (fluid balance) measurement.

In some example embodiments, a different group of hospital employees may be informed of a changing EWS at the various EWS threshold values. Also, in some example embodiments, the EWS threshold values may differ. In some other example embodiments, different score algorithms are used. For example, the score may be patient at risk score (PARS) or some other risk algorithm based on clinician observed information which is captured at a point of care. In some other example embodiments, a combination or two or more different score algorithms is used, such as, for example, a combination of the above described EWS algorithm and the PARS algorithm. In some further example embodiments, rather than using a risk score algorithm, such as ViEWS or PARS, a single vital sign is used as an indication of patient health deterioration, such as, pulse, temperature or blood pressure. However, using an algorithmically combined early warning score, such as ViEWS or PARS, has better specificity and sensitivity than a single trigger warning score, such as pulse or temperature. VIEWS is particularly advantageous because it has a high value of ‘area under the receiver-operating characteristics curve’ (AUROC). The AUROC value for ViEWS is at least 0.88. This attribute highlights that ViEWS is able to discriminate between survivors and non-survivors in a group of hospital admissions, such as a random group. It is therefore an advantage of the above-described example embodiment that it uses a score algorithm (also known as an aggregated weighted track and trigger system) having an AUROC of greater than or equal to 0.80, and in particular, having an AUROC value of greater than or equal to 0.88.

In the present example embodiment, EWS messages are sent to hospital employees, in accordance with FIGS. 5 and 6, when a patients health deteriorates below an EWS threshold value and then the patients health improves above an EWS threshold value. In some other example embodiments, EWS message may only be sent in one of these cases, for example, only when health deteriorates below a threshold but not when health improves above a threshold. In some example embodiments, an EWS messages are sent based on a single measurement of health, for example, when the patient is first admitted, the patient's health may be so poor that an EWS message is sent to a medical practitioner straight away. In this case, no deterioration is observed because the patients health status is measured for the first time. However, the operation of the system in responding to the poor status of health is the same as when responding to deteriorating health.

In the system 2 of FIG. 2, the score engine 14 communicates with the list manager 12 via the server 10. However, in some other example embodiments, the score engine 14 may communicate directly with the list manager 12 in addition to, or instead of, communicating via the server 10. Furthermore, in some example embodiments the score engine may communicate with analysis devices via the server instead of, or in addition to, communicating with analysis devices directly. In other words, the server may send the EWS messages.

### List Manager Operation

As mentioned above, in order to send messages, such as EWS messages, relating to particular patients it is necessary to determine which medical practitioners should be sent the messages, i.e. it is necessary to determine which medical practitioners are responsible for particular patients. The present example embodiment uses the list manager 12 to identify which medical practitioners are responsible for a given patient.

A doctor sets up one or a number of patient lists, where each patient list contains the patients the doctor is responsible for at any one time. These lists can correspond to a doctor's possible ‘roles’. For example, a junior doctor may work for a particular consultant on one shift, on a particular ward on other another shift, and cover an particular area of the hospital on a night shift. Typically, a list comprises one or more of the following: the patients on a particular consultant's list (the consultant/patient relationship being maintained by the measuring and analysis devices, as described above); the patients on a particular ward or number of wards; and, individual patients (for example, those added by the doctor using the analysis device). Therefore, the doctor can use the analysis device and the list manager to view a group of patients in a particular patient list, wherein that group may correspond to a group of patients located on a particular ward.

Also as mentioned above, the doctor can use the analysis device to select one or more of their lists as ‘active’. The ‘active’ list corresponds to the doctor's current ‘role’. A doctor will only receive messages, such as EWS messages, in respect of patients that are part of the doctor's active list. Therefore, the analysis device to which the doctor is logged-in only receives messages related to patients in an active list of the doctor.

Lists can also be managed and maintained using the interface of the base station 4. Specifically, the doctor may log into their account directly on the base station 4 using the interface, rather than logging into an analysis device. Once logged into the base station 4, the interface displays a ‘My List’ screen showing all the lists related to the doctor. For example, all public lists stored on the list manager 12 will be displayed, as well as any private lists created by the doctor. As mentioned above, one of the lists is designated active, and this represents the doctor's on duty list. The active list represents the list of patients that the doctor is currently looking after, i.e. the doctor's current ‘role’. The active list is the default list of patients displayed on an analysis device after the doctor has logged in. The doctor will receive messages sent in respect of each patient in the active list. The following describes the functionality of the ‘My List’ screen in managing and maintaining patient lists.

The interface allows the doctor to remove or add an active list designation to any list displayed in the ‘My List’ screen. Additionally, the interface allows the doctor to select anyone of the displayed lists, for example, by selecting a list using a mouse of the interface. Once a list is selected, the interface displays information relating to the selected list, such as, for example, the patients included in the list, the patients excluded from the list, the list creator, and the last person to modify the list. Patients may be included or excluded on an individual basis or a group basis. For example, all patients under the care of a particular consultant may be included, whereas one particular patient may be excluded.

The interface allows the doctor to remove a particular list from his ‘My List’ screen. It is noted that ‘removal’ is different from ‘deletion’ which is discussed below. If the doctor selects a list for removal, the interface no longer displays the list in the doctor's ‘My List’ screen. In some example embodiments, the interface only permits the doctor to remove particular lists, such as, for example, public lists.

The interface allows the doctor to edit a particular list displayed in his ‘My List’ screen. Specifically, the interface allows the doctor to modify list attributes, including: the list name; the list type (public or private); whether or not others are allowed to modify the list; the patients included in the list; and, the patient excluded from the list. As mentioned above, patients can be included or excluded individually or as part of a group, such as, for example, all patients in the care of a particular consultant, or all patients in a particular ward. In some example embodiments, patients may only be included or excluded individually or as part of a group. In some other example embodiments, patients may be handled differently depending on if they are being included or excluded.

The interface allows the doctor to delete a particular list displayed in his ‘My List’ screen. If the doctor selects a list for deletion, the interface no longer displays this list in the doctor's ‘My List’ screen and deletes it from the list manager. In some example embodiments, the doctor is only permitted to delete particular lists, such as, for example, the doctor's own private lists. Additionally or alternatively, the list manager may prevent the doctor deleting any public lists which are is use by another doctor, for example, as their active list. For example, the list manager may cause the interface to display a message notifying the doctor that the list cannot be deleted because particular other doctors are using it.

The interface allows the doctor to add a public list to their ‘My List’ screen. Specifically, a soft-key is displayed on the interface display which, when activated, displays a list containing all the public lists available to the doctor for inclusion in their ‘My List’ screen. Each public list displayed may be viewed, as described above, or added to the doctor's public lists. The interface also allows the doctor to create a new list. Specifically, a soft-key is displayed on the interface display which, when activated, permits the doctor to specify attributes of a new list. For example, these attributes may include: the list name; the list type (public or private); whether or not others are allowed to modify the list; the patients included in the list; and, the patient excluded from the list.

The list manager ensures that doctors' lists are kept up to date using the following mechanisms. Using the measuring device, the nurse will continually ensure the patient's consultant is recorded accurately because this information is captured during the clinical process of monitoring the patient's vital signs. Also, a doctor can add an individual patient to, or remove an individual patient from, one or more of their lists. This will mainly be done using the doctor's analysis device, however, it can also be done via the interface of the base station 4.

The list manager monitors the patient lists and ensures that every patient is on at least one active patient list. This is important because if a patient is not on any active lists no doctor to receive messages, such as EWS messages, if their health starts to deteriorate. In the present example embodiment, the list manager sends an alert message to hospital administrators if it determines that a patient is not on an active list.

When a doctor makes a new patient list their active list, they are effectively taking responsibility for a new set of patients. The doctor handing over the list can use the list manager to assign actions and send messages to the doctor taking over responsibility for the patients. The list manager can ensure that the new doctor sees these messages and actions when they designate a list as active. Accordingly, the list manager combines handover information with messaging and workflow functionality.

Another function of the base station interface is to display views which show hospital staff, such as administrative staff, which doctors are looking after which patients. This information can be used, for example, by a switchboard to direct calls to the correct clinician, or a nurse could easily find out which doctor or doctors to contact regarding the patient.

Furthermore, in order that auditing of changes to patient lists can be performed, the list manager logs and stores the following events: active list setting, new patient list creation, existing patient list modification and deletion, addition and removal of lists from particular doctor's ‘My List’ screen. In each case, data relating to the event must be stored, including, for example, the name of the user performing the event, the date and time of the event, and details of any changes that have been made.

A further advantage is that multiple copies of patient records and patient lists can be obtained from multiple locations. Such an arrangement is advantageous over a paper-based system wherein, for example, only one complete patient record is available from only one location.

### Analysis Device Software Application

Annex A provides a functional specification for a software application arranged to control the operation of the analysis device 8, in accordance with a variation of the above-described example embodiment. The software application comprises a user interface of the analysis device, which is displayed on the display of the analysis device. The software application is stored on the system partition 82 of the analysis device 8, however, it is to be understood that in some other example embodiments the application could be stored in a different portion of the long-term storage 78. In general, the software application controls the operation of the analysis device 8 so that it can provide at least some of the above-described functionality.

### Advantages of the Example Embodiments

The above-described system for monitoring patient health and notifying relevant medical practitioners when patient health deteriorates involves a number of different operations. Firstly, vital sign data is captured using a measuring device 6 at the point of care, such as the patient's bedside. The recorded data is transferred from the measuring device to the base-station 4 for storage on the server 10, and analysis by the score engine 14. The server 10 stores patient data so that data relating to each individual patient is retrievable by other system elements. The score engine 14 analyses data relating to individual patients by calculating a score, such as an EWS. The score provides an indication of whether the patients health is deteriorating or improving. Depending on the score value, the score engine determines whether medical practitioners, such as doctors or consultants, need to be notified because one of the patients require treatment. If the score engine determines that notification is necessary, the score engine consults the list manager 12 to identify which medical practitioners are responsible for the patient at the present time. The score engine then sends messages, such as an EWS message, to the medical practitioners responsible via their analysis device 8. On receipt of one of the messages, the first medical practitioner to react indicates to any other notified medical practitioners that they are en-route to the patient. Once the first medical practitioner has administered treatment to the patient, he notifies the other notified practitioners with details of the treatment.

An advantage of the above-described example embodiments is that medical practitioners, such as nurses, are instructed to take vital sign measurements. Specifically, once a nurse is logged into their measuring device, it is apparent which of their patients are overdue observations, and when observations are due for their other patients. This operation minimises hospital deaths caused by no observations being made for a prolonged period prior to a patient's death, or changes in vital signs not being detected. Furthermore, since vital signs are recorded at the point of care, they are input to the system immediately, therefore minimising the risk of errors in transcription.

Another advantage is that recognition of deterioration is automatically performed. Specifically, vital sign measurements are automatically transmitted to the server 10, and the score engine 14 automatically calculates a score based on those measurements. Since these operations are performed electronically, the chance of introducing human errors when calculating the score is minimised. Additionally, the score engine automatically sends messages according to its internal score thresholds. Further, the list manager enables identification of which medical practitioners are responsible for which patients, i.e. which medical practitioners should be sent messages. This operation minimises deaths attributed to there being no recognition of deteriorating health, or no actions undertaken, despite recordings of vital signs being performed.

A further advantage is that when a medical practitioner responds to a message sent in respect of one of their patients, the practitioner must first notify any other practitioners that he is responding. According to this operation, the other practitioners do not waste their time responding when it is not necessary. Additionally, once the responding practitioner has responded, he must notify the other practitioners with details of the intervention. Therefore, up-to-date patient information is quickly distributed around those who require it most urgently. Further, details of which doctor responded, and what their response was, is stored for auditing purposes, such as, for example, investigating a patient's death.

### Second Example Embodiment

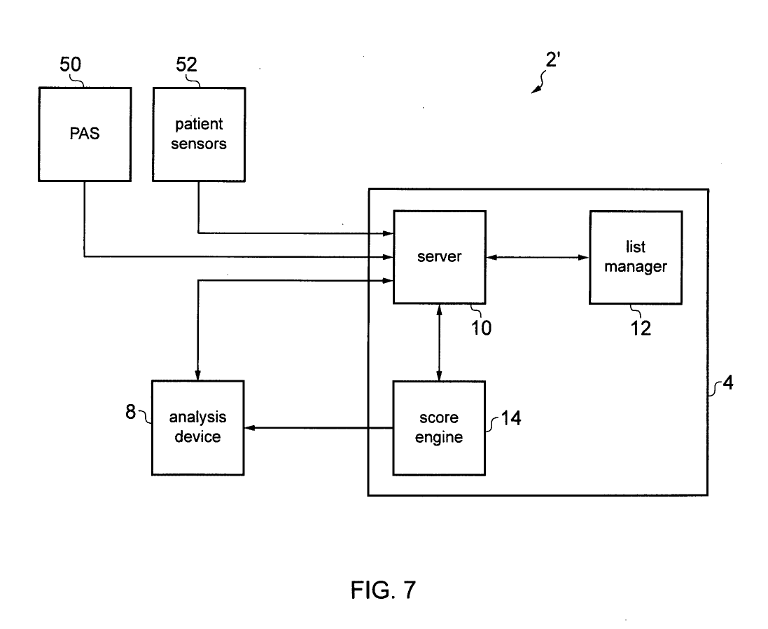

FIG. 7 is analogous to FIG. 1, and illustrates a modification to the above-described system 2 to form a system 2′. The differences between the system 2 and the system 2′ are as follows.

In the arrangement of FIG. 1, vital signs of a patient are measured and recorded by a hospital employee, such as a nurse, using a measuring device 6. The recorded vital signs are then transmitted from the measuring device 6 to the server 10. In the arrangement of FIG. 7, a patient's vital signs are obtained from a combination of two sources. The first source is the patient administration system (PAS) 50. The PAS 50 is a clerical system used in the hospital to keep an up-to-date record of each patient. Each record includes basic patient information, such as contact details, as well as some medical information, such as test results and observation results. The PAS 50 is in communication with the server 10 such that the patient records on the server 10 can be updated by the PAS 50.

The second source is one or more sensors 52 connected to each patient and arranged to automatically record vital signs from the patient. For example, a patient may be connected to a sensor capable of measuring their temperature, pulse, or blood pressure. The one or more sensors 52 are in communication with the server 10 such that the patient records on the server 10 can be automatically updated by the sensors 52.

The operation of the arrangement of FIG. 7 is similar to the above-described operation of the arrangement of FIG. 1. However, vital sign recordings for a patient are retrieved electronically from the sensors 52 and the PAS 50. Accordingly, there is no need for vital signs to be specifically recorded via a measuring device. It is advantageous to combine sensors 52 with PAS 50 because it may not be possible to provide a sensor to measure each vital sign necessary for calculation of the EWS, or similar score. For example, it may not be possible to measure the neurological indicator AVPU using an automatic sensor. It is also advantageous because the PAS 50 may not always be up-to-date. For example, the PAS 50 may only be updated by clerical staff who only work between 09:00 and 17:00, Monday to Friday.

It is an advantage of the arrangement of FIG. 7 that it is not necessary for a hospital employee to specifically measure and record vital signs for each patient. It is a further advantage that measurements taken using the sensors 52 are received electronically directly from the sensor. Accordingly, there is less need to take manually take vital sign measurements. Additionally, there is less chance that errors, such as errors in transcription, will be introduced. Further, measurements can be taken using the sensors 52 at any time and with any frequency, without causing any additional burden on hospital employees.

It is to be understood that in some example embodiments only one of the PAS 50 and the sensors 52 is used for collecting vital signs. In some other example embodiments, a combination of PAS 50 and sensors 52 is used some of the time, and only one of the PAS 50 and sensors 52 is used for the rest of the time. Additionally, in some further example embodiments, measuring device recordal, as described with reference to FIG. 1, may be incorporated into anyone of the example embodiments described with reference to FIG. 7.

### Third Example Embodiment

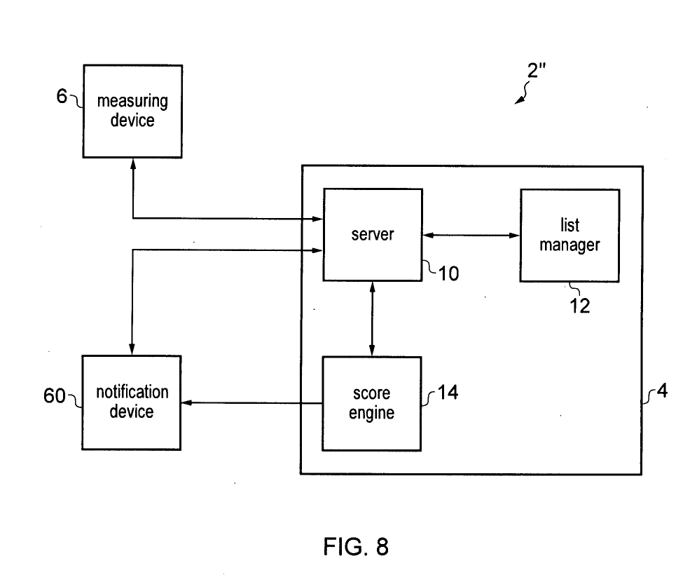

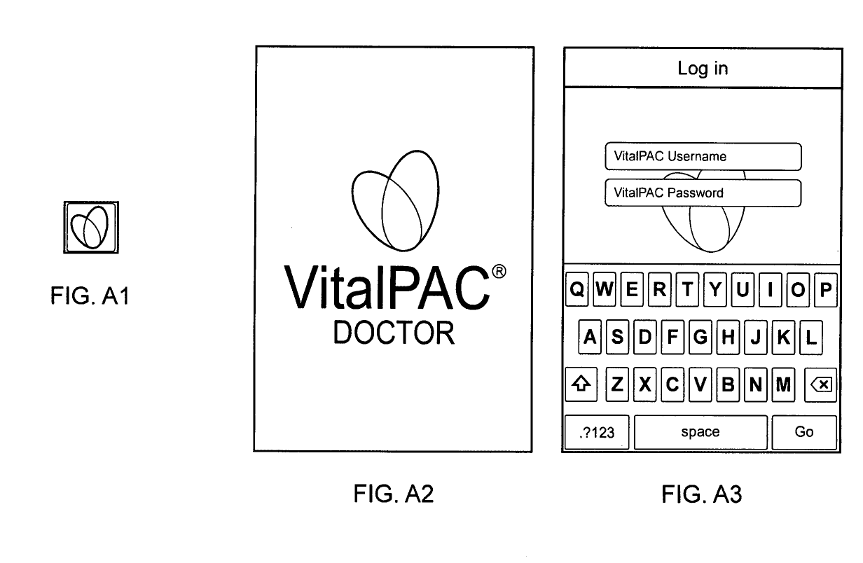

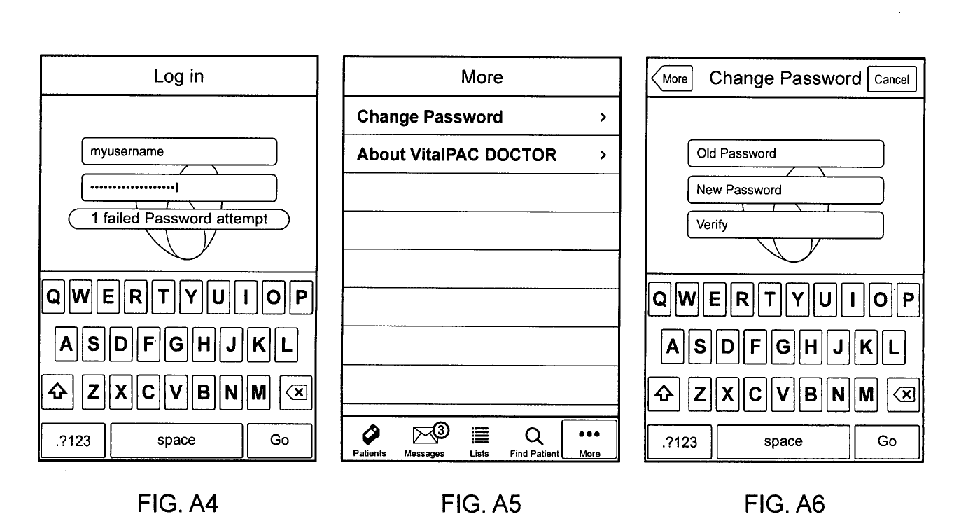

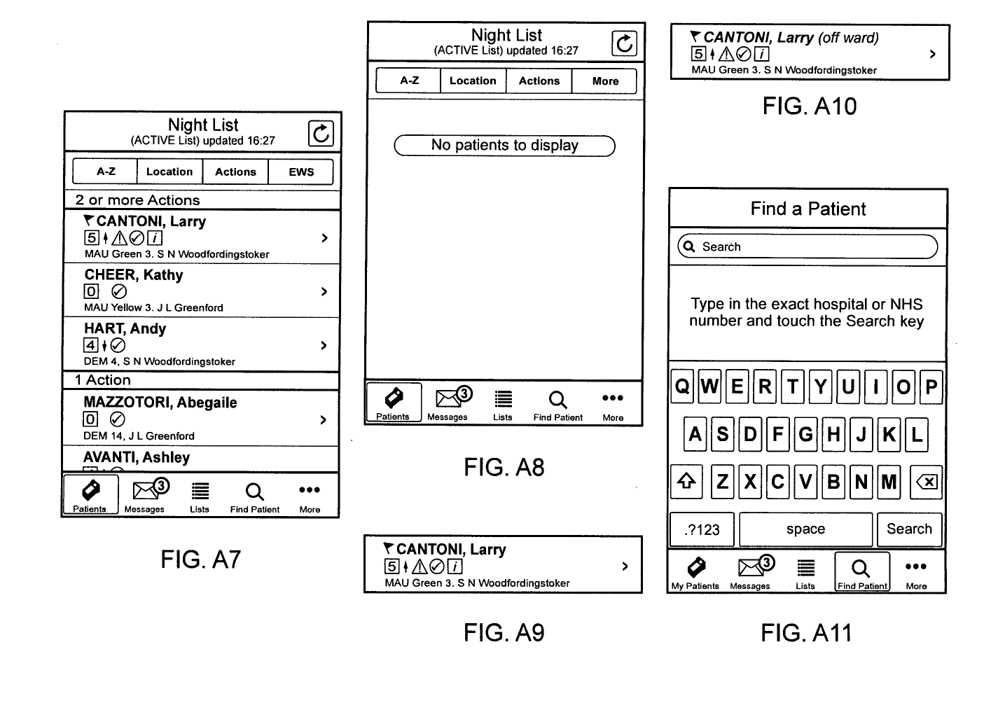

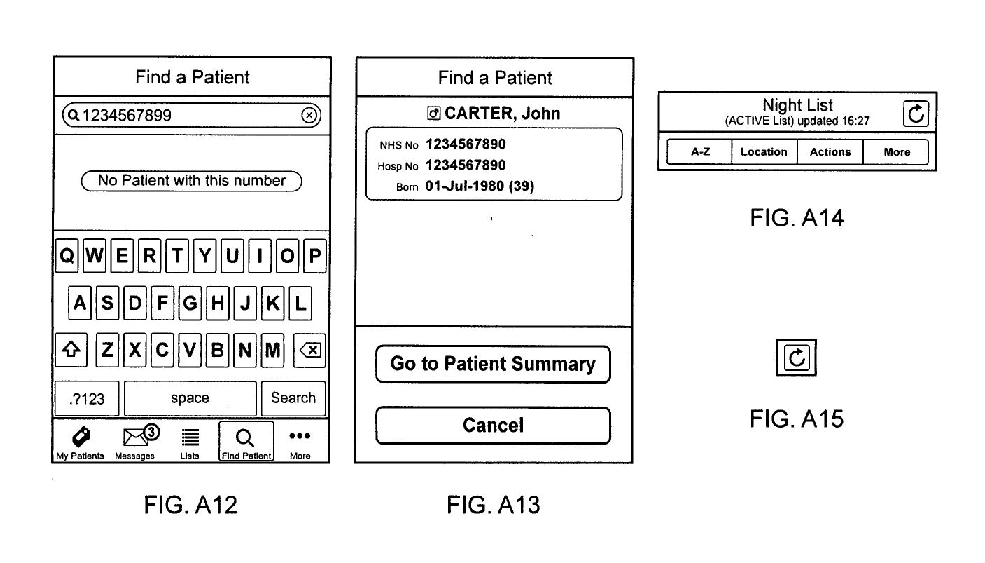

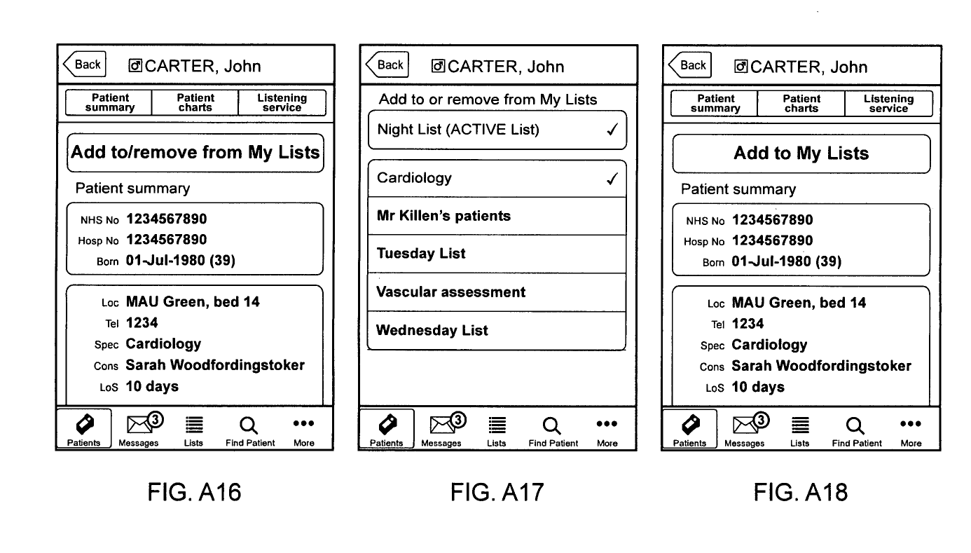

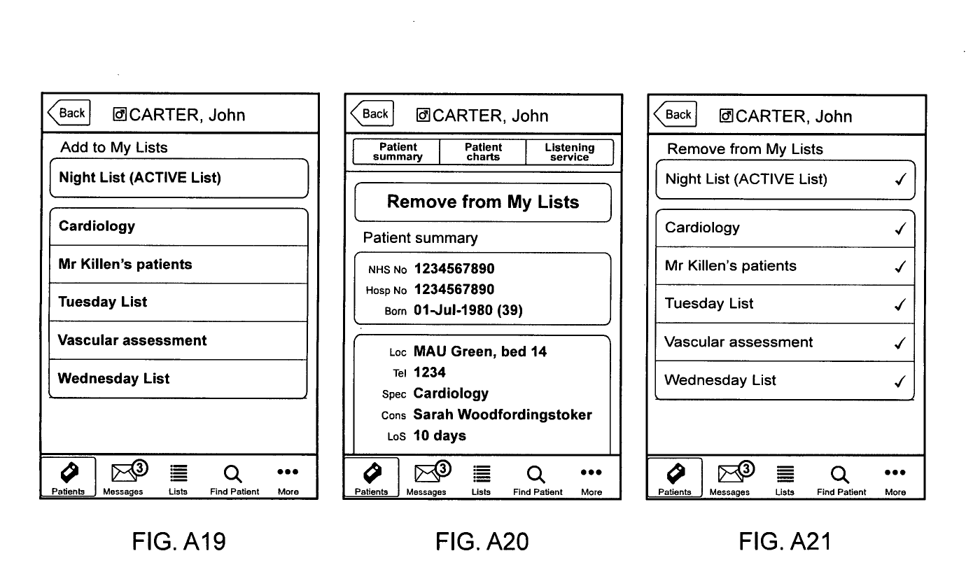

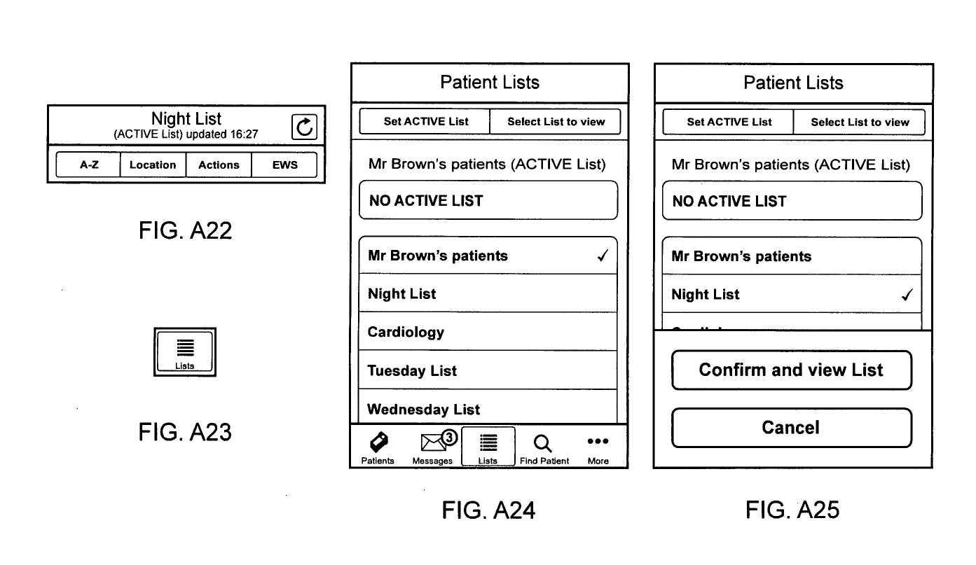

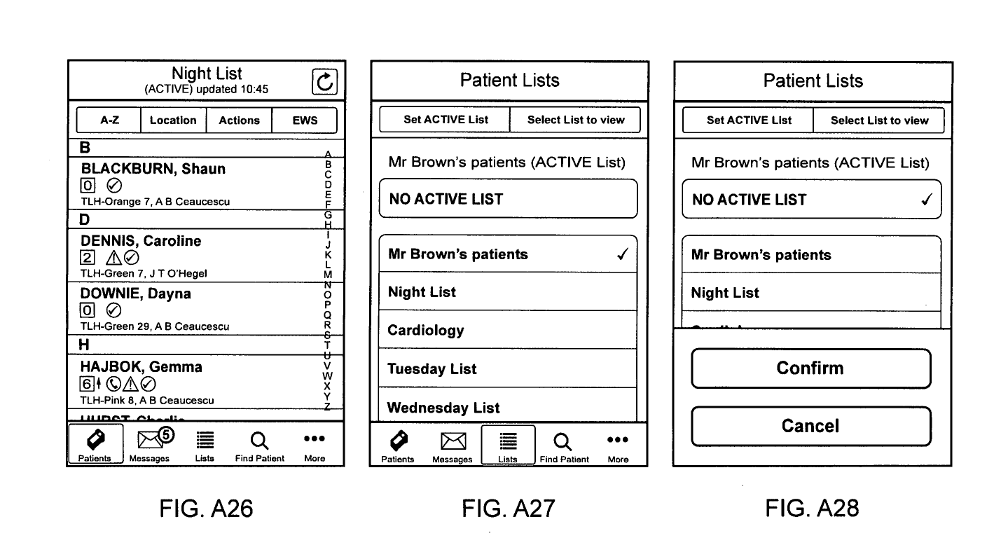

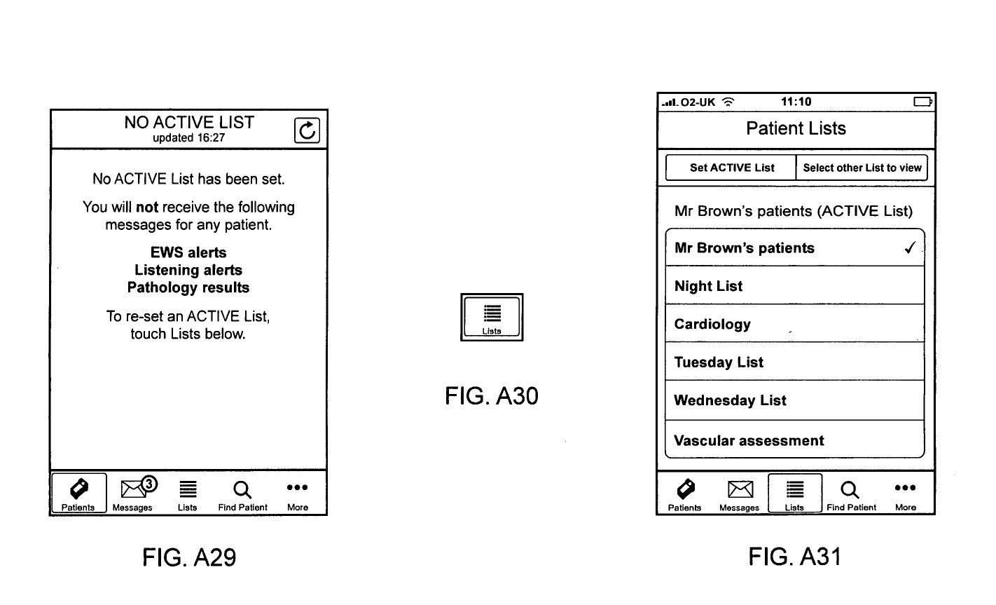

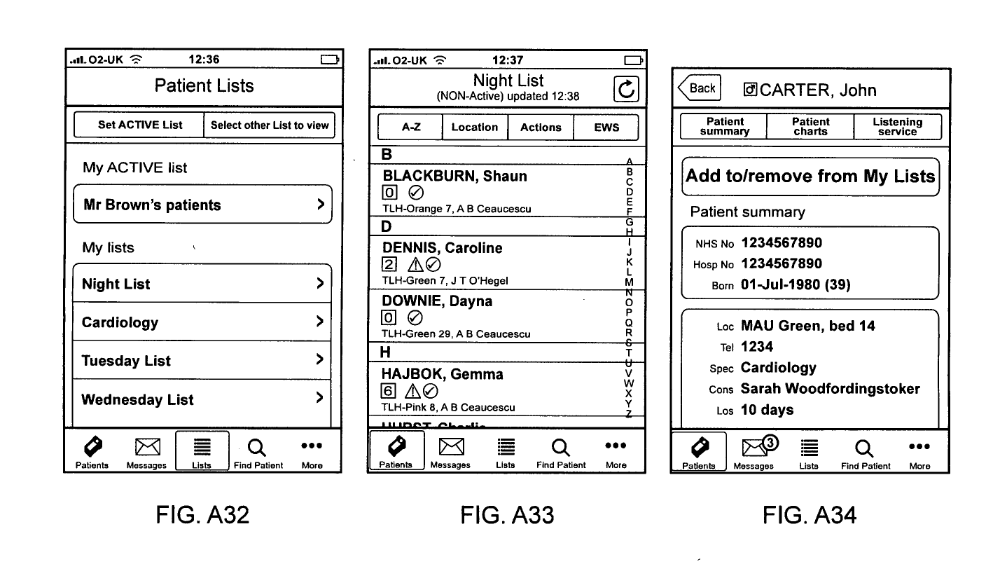

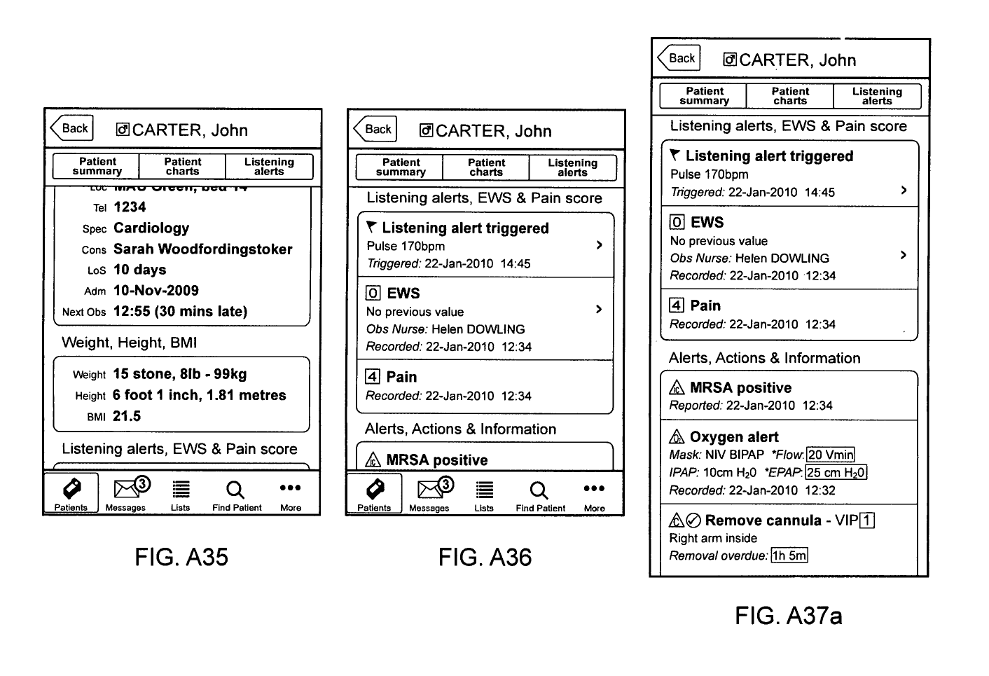

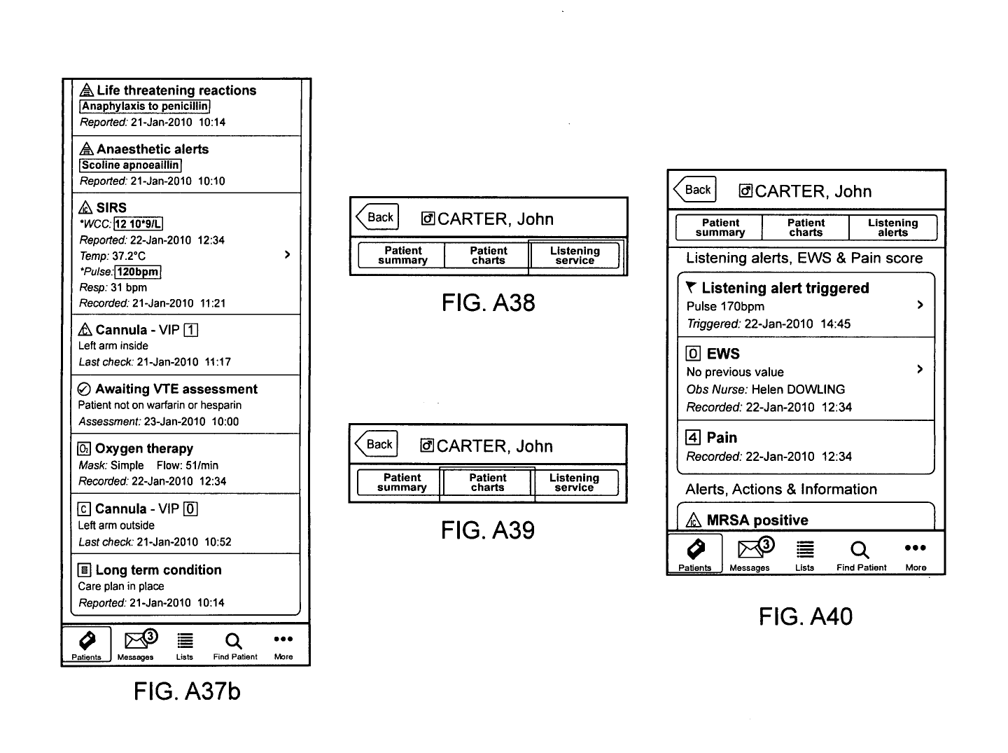

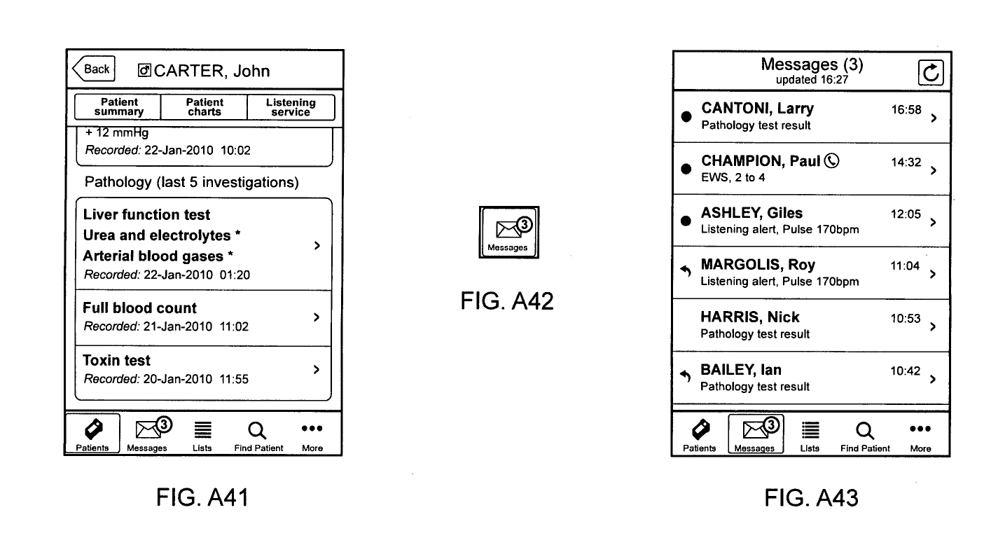

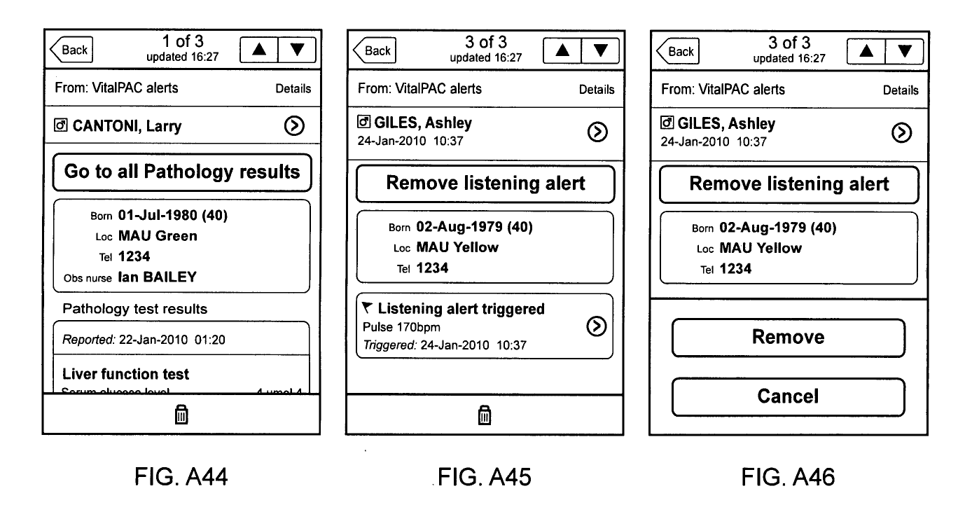

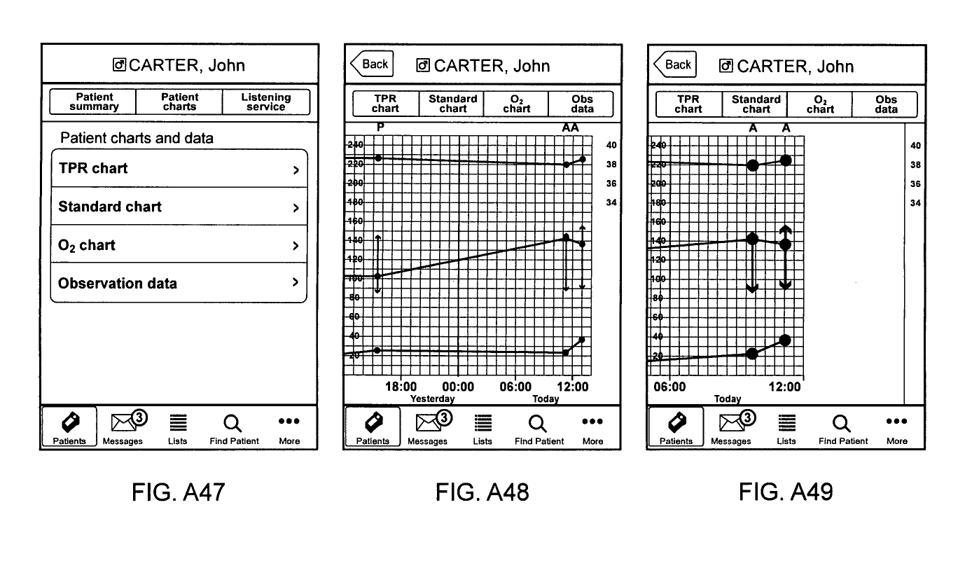

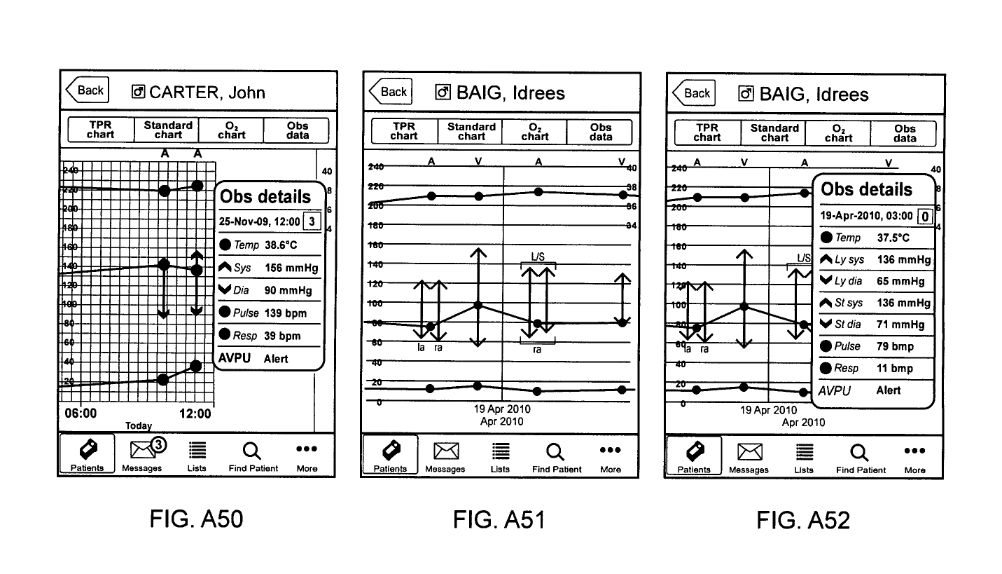

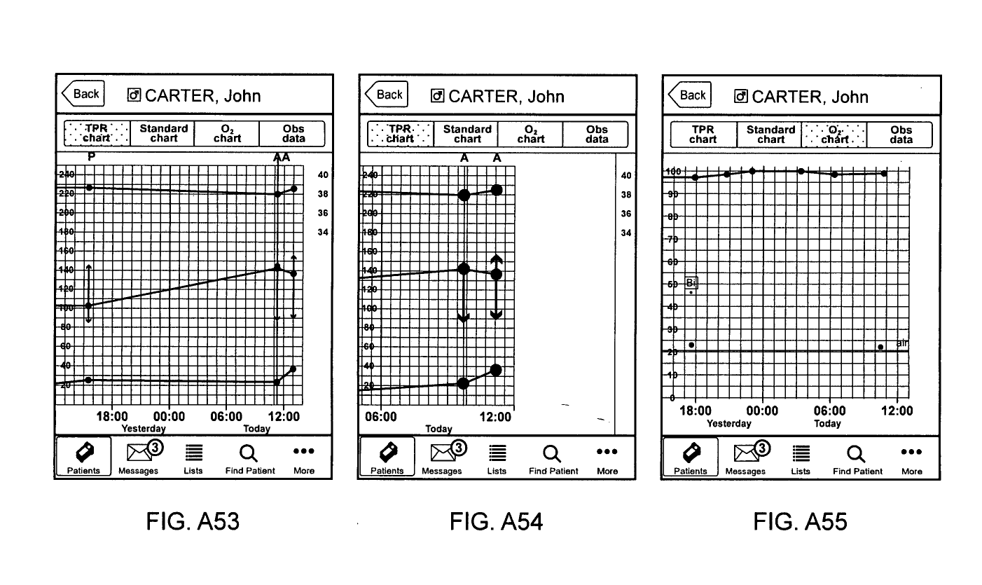

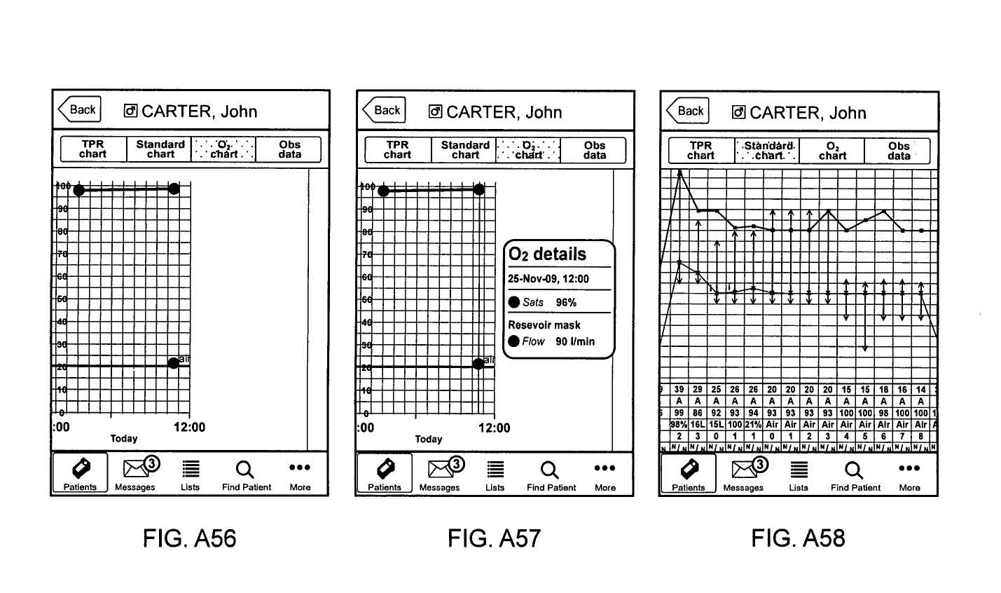

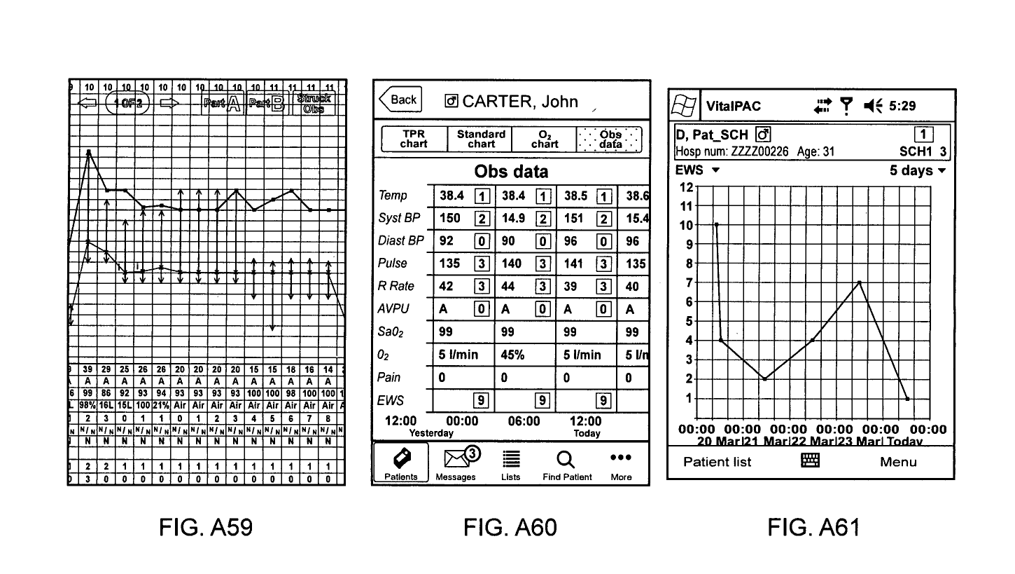

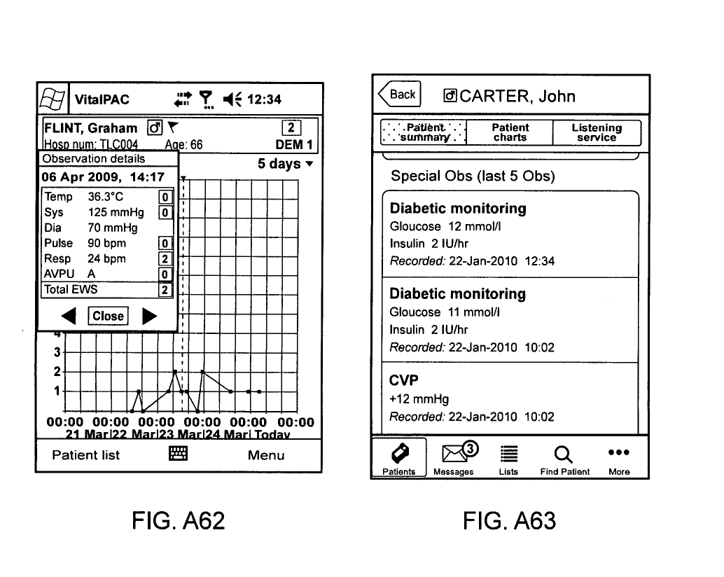

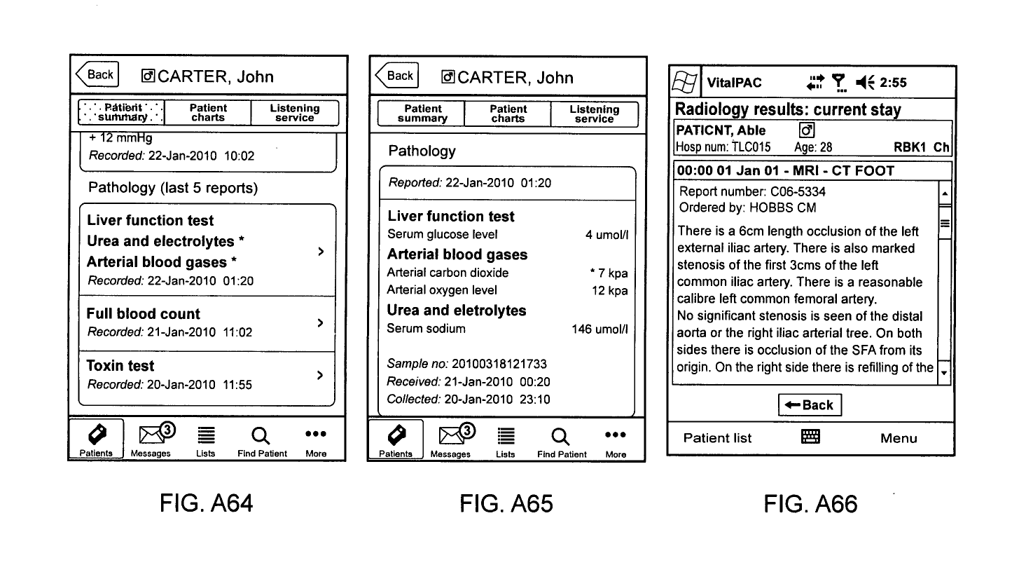

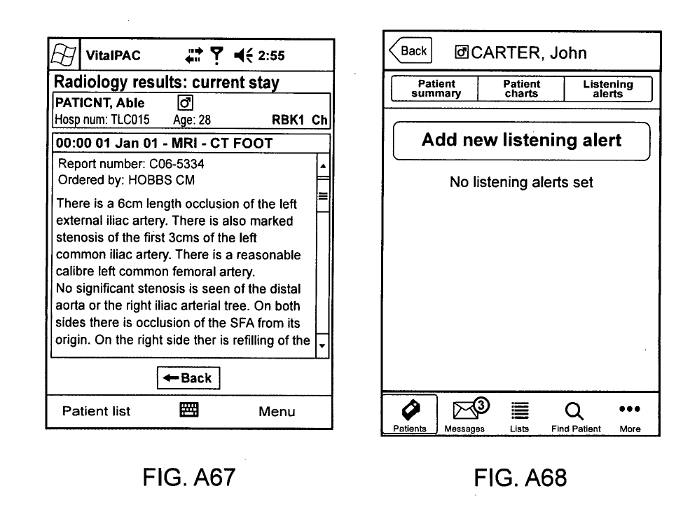

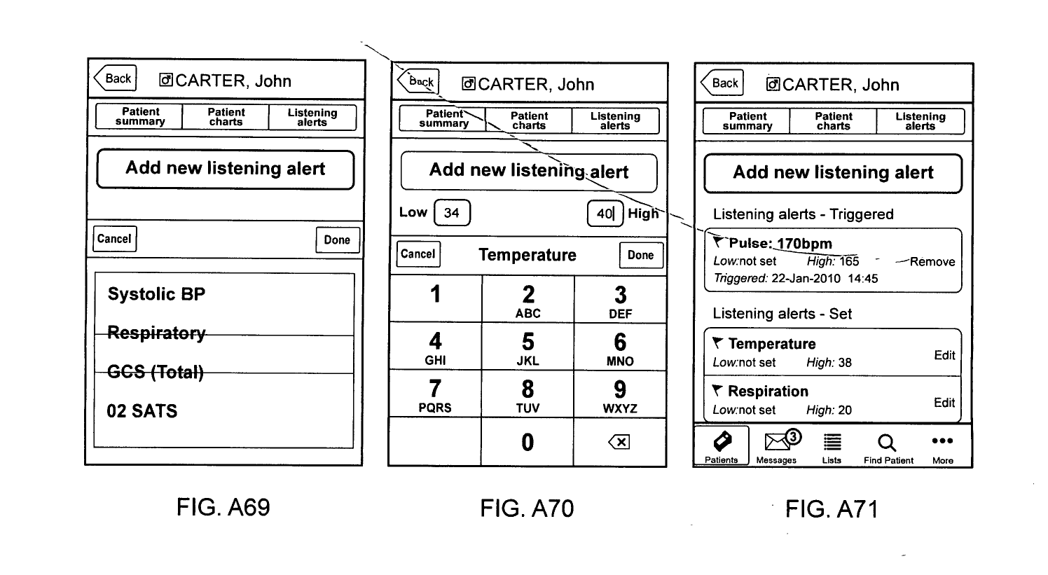

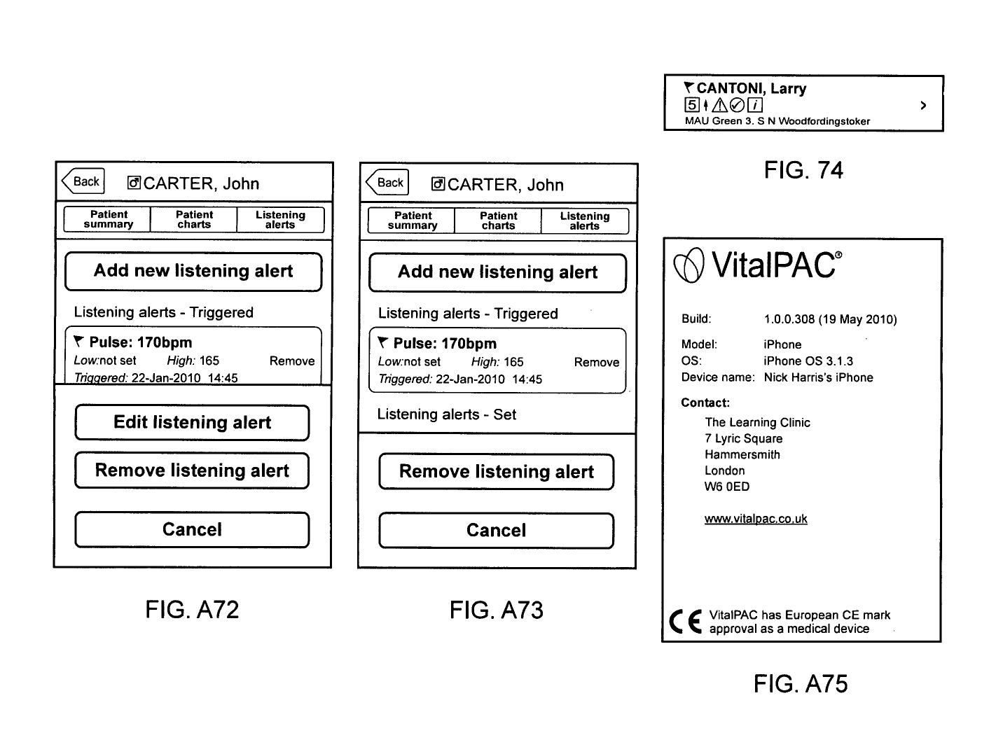

FIG. 8 is analogous to FIGS. 1 and 7, and illustrates a modification to the above-described systems 2 or 2′ to form a new system 2″. The differences between the systems 2 and 2′ and the system 2″ are as follows.

In the systems 2 and 2′, messages relating to patients, such as EWS messages, are sent to medical practitioners, such as a doctor, via the doctor's analysis device 8. In the system 2″, one or more analysis devices 8 are replaced by a notification device 60. The notification device 60 is capable of notifying the doctor when new information is available for one of their patients. For example, the notification device 60 may be a pager or beeper. As well as notifying the doctor that new information is available, the notification device may provide some additional information, such as: an identifier of the patient for which new information is available, the nature of the new information, a telephone number to call to discover more about the new information. On receipt of the notification, the doctor can investigate further to discover more about the reason for his notification. For example, the doctor may log onto the base station interface to discover more about the notification.

An advantage of the system 2″ is that it is simple. For example, a notification device can be a simple device which can be smaller and cheaper to produce. Stated differently, the notification device can have a reduced capability to the analysis device.

It is to be understood that the system 2″ can be combined with any variation of the above-described systems 2 or 2′. For example, the measuring device of FIG. 8 may be replaced with the PAS and/or sensors of FIG. 7.

Various modifications and improvements to the above-described example embodiments may be apparent to the skilled person when reading the above disclosure, any and all of which are intended to be included within the scope of the appended claims. For example, the above-described example embodiments have been described with reference to patients in a hospital. However, some other example embodiments could operate in other environments, such as, disaster recovery camps or any other area where the monitoring people's health within the area is required. Also, some other example embodiments could be used with animal patients rather than human patients and, therefore, some other example embodiments could be applicable to animal hospitals.

In the above-described example embodiments, the emphasis has been on detecting deteriorating health of a patient. However, in some other example embodiments, the system could be principally concerned with detecting unchanged or improving health of patients. Further, in some example embodiments, rather than identifying changing health of a patient, the system is arranged to identify the status of a patient's health, i.e. an absolute measure of the patient's health at one point in time. For example, in some embodiments, when a patient is first admitted to hospital, the status of the patient's health is identified and messages are sent to one or more medical practitioners responsible for, or associated with, the patient if the health status is below a particular value or threshold. According to this operation, the doctor is notified of ill health (i.e. an absolute indicator) rather than deteriorating health (i.e. a change indicator). In some example embodiments, messages are sent in dependence on the status of a patient's health, as well as, in dependence on deteriorating patient health.

In the above-described example embodiments, the system is used by hospital employees, including nurses, doctors and consultants. However, in some other example embodiments, the system could be used by other personnel, for example, other medical personnel, or alternatively, non-medical personnel, such as, administrative personnel.

In the above-described example embodiments, the system comprises a thick server in the form of the base-station, and thin clients in the form of the measuring device and the analysis device. However, it is to be understood that alternative arrangements are equally valid and within the scope of the appended claims. For example, the measuring device and analysis device could be thick clients, whereas the base-station could be a thin server. Specifically, in some example embodiments the measuring device could include one of the server, the score engine and the list manager. In these cases, the base-station may include the others of the server, the score engine and the list manager. In some other example embodiments, the measuring device could include two, or all, of the server, the score engine and the list manager. Further, in some other example embodiments, a single hand-held mobile computing device could provide each system element, i.e. the measuring device, the analysis device and the base-station. Alternatively, a single hand-held mobile computing device could provide only some of the system elements, for example, a mobile device could provide all system elements apart from the server and the list manager, which could be provided in one or more separate elements, such as a base-station. Furthermore, some example embodiments may combine any or all of these different arrangements. It is to be understood that in each of these alternative arrangements, the elements of the system are capable of communicating with each other to transfer data, such as patient information or patient list information, so that the system as a whole can perform the above-described operations.

It is described above how messages are sent in response to the health of a patient. For example, messages may be sent in dependence on the status of the patient's health, or on deterioration of the patient's health. It is to be understood that in some example embodiments, additional messages are sent which relate to the patient but are not necessarily triggered by a measure of the patient's health. For example, additional messages may be sent in respect of the patient when new test results become available for the patient, such as pathology results. Specifically, on receipt of new test results at, for example, the server, the list manager identifies which medical practitioners are associated with the patient corresponding to the new test results. The system then sends a message concerning the new test results to the associated medical practitioners. The message may contain the test results, a summary of the results, or an indication that they are available. According to this operation, the advantages of the list manager may be obtained without necessarily requiring the use of the score engine. In particular, the system can be used to identify which medical practitioners are associated with a patient, without requiring that a score is calculated for the patient or that a score message is sent in respect of the patient.

In the above-described example embodiments, a variety of soft-keys are described to enable functions of the various system elements. It is to be understood that one or more of the above-described soft-keys may be substituted by a hard-key, or some other activation means, such as, a voice command received by a microphone of the analysis device.

### ANNEX A

### 2—Detailed Functional Requirements

### 2.1—Login

| | |
|---|---|
| 2.1.1 | The application will be represented on the home screen by the icon of FIG. |
| | A1. Tapping on this icon will launch the application. |
| | Whilst the application is initializing the splash screen of FIG. A2 will be |
| | shown. |
| 2.1.2 | Once initialization is complete the login screen of FIG. A3 will be displayed. |
| | A valid username and password must be entered before the user can proceed. |
| | If an invalid username or password is entered the screen of FIG. A4 will be |
| | displayed. |
| | (Note: For the demo release, any user name and password will be accepted.) |
| 2.1.3 | If no activity (screen tap) is registered, an “Auto-Lock” function will lock the |
| | screen in the time period determined by the user's settings. Passcode locking |
| | of a device within a Hospital or Trust environment, is recommended. |
| | (Note: the application will have no bespoke time out function.) |
| 2.1.4 | There shall be no support for “Check for updates” in the demo release. |
| 2.1.5 | The user shall have the option to change their password via the More tab |
| | shown in the screen of FIG. A5. When selecting this option the user will first |
| | be prompted to enter their existing password via the screen of FIG. A6. Then |
| | the user will be prompted for their new password and asked to confirm it. If it |
| | is successfully saved a confirmation message will be displayed. Similarly an |
| | error message will be displayed if the password fails to save, with an |
| | explanation of why. |
| | (Note: In the demo version, any changes to the password will be discarded |
| | when the application is exited.) |

### 2.2—Logout

| | |
|---|---|
| 2.2.1 | You can exit the application at any point by |
| | pressing the device's home button. |

### 2.3—Patient Management

After logging in the user will be taken directly to the Patient List view shown in the screen the screen of FIG. A7 of the Active list.

| 2.3.1 | The default list will be the current Active list. | |
|---|---|---|
| | At the Patient List screen, users will be able to filter (sort) the Active List by: | |
| | A-Z, Location, Actions, and early warning score (EWS). | |
| | The default sort order for patients is alphabetical, but within the following | |
| | parameters: | |
| | Location sort order is—Letter bar containing names of locations in | |
| | alpha-numeric order, followed by A-Z listing of patients; | |
| | Action sort order is—Letter bar containing up to three of the | |
| | following headings: 2 or more Actions; 1 Action; No Actions; each | |
| | followed by A-Z listing of patients. | |
| | EWS—To accommodate different Trusts wanting to use their own | |
| | Early Warning Systems (EWS, PARS, ViEWS etc), we need three | |
| | different configurable parameters within the code: Early warning | |
| | score; Risk level (low, medium, high, critical); and Name. This will | |
| | allow each Trust to configure the white, amber, red colours to their | |
| | own scoring system and allow the bespoke naming of their system | |
| | within the application. Within the Patient List, up to four letter bars | |
| | should be deployed, named: Critical, High, Medium, and Low. | |
| | If the currently viewed list is empty then the text ‘No patients to display’ | |
| | should be displayed, as shown in FIG. A8. | |
| | Note: this feature will not be required for the demo release. | |
| | (Note: Sorting shall not be cumulative e.g. the user can sort by location or | |
| | EWS not location and then EWS.) | |
| 2.3.2 | The patient list will contain the following fields (an example is shown in | |
| | FIG. A9). | |
| | Listening Alert (denoted by a flag. ‘ [symbol] ’ will be shown against the | |
| | patients name), see section 2.5.1 for more details. | |
| | This is followed by the patient's name displayed in the following way— | |
| | Capitalised patient last name followed by a comma, followed by the | |
| | patient's first name in upper and lower case text—e.g. LASTNAME, | |
| | Firstname. | |
| | This is the convention for displaying names that are too long to fit | |
| | on one line: | |
| | LONGLASTNAME, Asmuchofthefirstnameaspossibl . . . | |
| | VERYVERYVERYVERYVERYVERYLONGLASTNAME, A. | |
| | VERYVERYVERYVERYVERYVERYVERYLONGLAS . . . , A. | |
| | This is followed by icons, in the following order: | |
| | EWS (plus escalation arrow), Alerts, Actions, Information. | |
| | (Note: The EWS score shall only be shown if it is less than 30 | |
| | hours old) | |
| | Alerts, Action and Information. | |
| | The icons are followed by: Location (Ward Short Name, Bed Number | |
| | and Chairs & Trolleys), Comma, Consultant's name (formatted: A N | |
| | Lastname). | |
| | (Note: Bay names (if applicable) will not be shown.) | |
| 2.3.4 | An indication shall be given as to whether the patient's EWS is getting better | |
| | or worse since the last set of observations were taken by the use of arrows: | |
| | ↑↓. | |
| | The EWS score shall only be shown if it is less than 30 hours old. | |
| | The EWS score shall be colour coded according to the following table. | |
| | Severity Score | Colour |
| | Trust configurable | White background with black text |
| | Trust configurable | Yellow background with black text |
| | Trust configurable | Amber background with black text |
| | Trust configurable | Red background with white text |

| | |
|---|---|
| 2.3.5 | An indication shall be given if the patient is temporarily off-ward, by adding |
| | a grey overlay to the row, italicising the patient's name and adding the |
| | phrase (off ward), see FIG. A10. |
| 2.3.6 | The user will have the ability to be able to access the Patient Summary view |
| | by touching any part of the patient's name row. This extra content is denoted |
| | by the disclosure arrow, far centre right of the row. |
| 2.3.7 | The user shall have the ability to find a patient using their NHS or hospital |
| | number on the find screen, see FIG. A11. Partial numbers cannot be |
| | entered. |
| | If the patient isn't found then a message is displayed to inform the user that |
| | there were no matches for that hospital number or NHS number, see FIG. |
| | A12. |
| | (Note: that the display all NHS Numbers must comply with predefined |
| | formatting.) |
| | If the patient is found, a dialog is shown with the gender icon, patient name, |
| | and NHS number hospital number, and the patient's data of birth with the |
| | option to review the patient's data using the ‘Go to Patient Summary’ button, |
| | see FIG. A13. If the user selects ‘Cancel’ then the user is returned to the |
| | Find a patient screen. |
| 2.3.8 | An indication shall be given to the user for when the view was last updated, |
| | see FIG. A14. The maximum automatic refresh interval is 5 minutes |
| 2.3.9 | A Refresh button (see FIG. A15) can be used to ensure that the patient list |
| | is showing all the latest data. |
| | (Note: No Refresh functionality is required for the demo release.) |
| 2.3.10 | The user shall have the ability to add patients from any ward to any of their |
| | “My Lists”. |
| | On the Patient Summary view an “Add/Remove from my lists” button will |
| | be available. In the case where the patient is on some of the lists the screens |
| | may show as FIGS. A16 and A17. If the patient is not on any of the lists |
| | then the screens may show as FIGS. A18 and A19. If the patient is on all |
| | the lists then the screens may show as FIGS. A20 and A21. |

### 2.4—Patient Lists

The user shall be provided with the ability to look at and set ACTIVE, non-ACTIVE lists, as well as view other user's public lists and Ward lists.

The default list upon opening the application will be the currently
selected ACTIVE list. The ACTIVE list name is shown in the header
bar, together with the last time that the application refreshed
its data from the server, see FIG. A22.
(Note: For the demo release this value will always be the current time.)
If the user chooses to view a Ward list, then the Ward Name would
be shown in place of the list name.

### 2.4.1—Set ACTIVE List

| | |
|---|---|
| 1. | The default patient list to view upon opening the application, |
| | will be the ACTIVE List. |
| 2. | To set a different list as the ACTIVE List the user must touch |
| | the Lists tab, see FIG. A23. |
| 3. | Upon opening this page, the default view would be the “Set |
| | ACTIVE List” sort tab at the top of the screen, see FIG. A24. |
| 4. | To change the ACTIVE List, the user simply touches another List |
| | row. This then gets ticked and hi-lights in blue text, see FIG. A24. |
| 5. | Upon touching the new List row a dialogue appears and says: |
| | ‘Confirm and view List’ and ‘Cancel’, see FIG. A25. |
| 6. | If the user chooses “Confirm and view List”, they travel |
| | to the newly selected ACTIVE List, see FIG. A26. |

(Note:
The active list is the list that the user will receive messages and actions on)

### 2.4.2—Setting NO ACTIVE LIST

Clinician's will be able to set their analysis device so that they
receive no messages or alerts. This would be done by selecting “NO
ACTIVE LIST” while within their connected environment.
(NOTE: If the clinician sets their ACTIVE List to “NO ACTIVE LIST”
while not connected to the hospital server by WIFI, VPN, or other such
service, the app will continue to function in the same mode as it was
when last connected to the hospital server.)

| | |
|---|---|
| 1. | To set the ACTIVE List to “NO ACTIVE LIST”, the user must |
| | touch the “NO ACTIVE LIST” row, see FIG. A27. |
| 2. | Upon touching the row, the action sheet of FIG. A28 displays the |
| | following options: ‘Confirm’ and ‘Cancel’, see FIG. A28. |
| 3. | If the user chooses “Confirm”, they travel to an empty patient |
| | list screen containing a warning message, see FIG. A29. |
| 4. | To return to an ACTIVE List that will receive messages and alerts on, |
| | the user should repeat step as in 2.4.1. |

### 2.4.3—Selecting Another List to View

| | |
|---|---|
| 1. | The default patient list to view upon opening the application, will be the ACTIVE List. |
| 2. | If the user decides they want to look at another List, without changing the ACTIVE List, |
| | they must first touch the “Lists” tab, see FIG. A30. |
| 3. | The default view will be the “Set ACTIVE List” sort tab, so the user must then touch the |
| | “Select List to view” sort tab, see FIG. A31. |
| 4. | In this view the currently selected list to view will be highlighted by default, see FIG. A32. |
| 5. | As soon as the user touches another list, that row becomes highlighted and the user |
| | travels to the newly selected Patient List, see FIG. A33. |

(Note: Sort tab “Select other List to view”, should read “Select List to view”.)

### 2.5—Patient Summary

The user shall be provided with the ability to view a patient summary screen, giving a snapshot of the patient's condition. This summary view is be accessed by tapping anywhere in the patient's name row on the patient list.

(Note: In all cases where a date and time is shown, if the date is today then only the time need be shown however in all other cases the data and time must be displayed.)

| | |
|---|---|
| 2.5.1 | The Patient Summary view shall display information about the patient, see FIG. |
| | A34 to A36. This shall include: |
| | Patient summary |
| | Gender icon, |
| | Patient's Surname and first name, |
| | NHS Number, |
| | Hospital Number, |
| | Date of birth (and age). |
| | Other Patient details |
| | Patient Location - Ward [short code], Bay [short code], Bed/Chair/Trolley |
| | Ward phone extension number, |
| | Specialty, |
| | Consultant, |
| | Length of Stay, |
| | Admission date, |
| | Next Observations due. |
| | Weight, Height, BMI |
| | Patient's weight in Stone, lbs - kg, |
| | Patient's height in Feet and inches - metres. |
| | Listening alerts, EWS & Pain score |
| | Listening alerts. If there are any listening alerts set or triggered, then it shall be possible |
| | to tap on a link to take the user to the Listening alert summary view as specified in |
| | requirement 2.9. |
| | The latest EWS score (with ↓↑), the EWS score shall only be shown if it is less than 30 |
| | hours old. Note: there are two up and down arrow states to differentiate between a small |
| | change i.e. 1 EWS point, and a larger change i.e. >=2 EWS points. This screen links to |
| | Tabulated Observation data, |
| | The last observation nurse, |
| | The Pain score for the patient. |
| | These are followed by alert icons set for the patient. The order for these icons is: |
| | Alerts, Actions, & Information |
| | Red triangles (any associated action icons are placed beside these alerts), |
| | Amber triangles, |
| | Action circles, |
| | Information squares. |
| | Information shown is in the following order: |
| | Icon (or icons) |
| | Name of alert, action or information |
| | In the case of Cannula, this name should be followed by a VIP score, if available |
| | Information about the condition (see below) |
| | Date and time (see below) |
| | a) MRSA (red) alert |
| | Reported date and time |
| | b) Oxygen (red) alert |
| | Mask type Flow rate/concentration |
| | Recorded date and time |
| | c) (Red) Remove Cannula + VIP score |
| | Site of Cannula |
| | Removal overdue time in days, hours and minutes |
| | d) Other red alerts |
| | Information |
| | Reported date and time |
| | e) SIRS (amber) alert |
| | Pathology data |
| | Recorded date and time (could apply to more than one set of data) |
| | f) (Amber) Cannula + VIP score |
| | Site of Cannula |
| | Last check date and time |
| | g) (Red, Amber or blue) Action icon (if on its own) |
| | Information about required action |
| | Date and time action is required, imminent or overdue |
| | h) Oxygen (square information icon) |
| | Mask type Flow rate/concentration |
| | Recorded date and time |
| | i) Cannula (square information icon) |
| | Site of Cannula |
| | Last check date and time |
| | j) Other (square information icons) |
| | Information about the condition |
| | Reported date and time |
| | An example of the maximum number of observation alerts is provided by FIGS. A37a and |
| | A37b. |
| 2.5.2 | This requirement shows how the user can navigate to the other screens that contain patient |
| | data. |
| | Listening alerts, see FIG. A38 (see section 2.9 for more details). |
| | Patient Charts and Standard Observations, see FIG. A39 (see section 2.7 for more |
| | details). |
| | Observation data, see FIG. A40 (see section 2.7 for more details). |
| | Pathology, see FIG. A41 (see section 2.8.1 for more details). |

### 2.6—Messages

The view shows a list of all the messages that have been sent to the user.

| | |
|---|---|
| 2.6.2 | The user will be able to navigate to the messages list from the patient list by tapping |
| | on the messages tab, see FIG. A42. |
| 2.6.3 | Messages which have not been read or acted upon will be identified by a blue dot, as |
| | unread emails in the analysis device's email client, see FIG. A43. A refresh button |
| | shall be made available to allow the user to check for new messages. When refresh |
| | is tapped a standard refresh animation shall be shown in the top left corner of the |
| | screen. |
| 2.6.4 | Tapping on a message row will reveal the message details. Depending on the |
| | message type, different options will be available. In each case however the |
| | following functionality/information will be present (as shown in the example of FIG. |
| | A44): |
| | Identification of the message sender. |
| | “Details” reveals other people the message was sent to. This blue word changes to |
| | Hide to allow the user to hide this information. |
| | The title of the email. This will always be the name of the patient the |
| | message refers to. |
| | Big navigational button (see variations of FIGS. A45 and A46). |
| | Other details including (if known): Birth date and age, Ward location, Ward |
| | extension number. |
| | Message content - this will vary according to the type of message sent i.e. |
| | pathology results, listening alert or EWS score. This is followed by the time the |
| | message was triggered or reported (this information is the same as the message |
| | sent time). |
| | Touching the blue disclosure arrow beside the patient's name will take the user to |
| | the top of the Patient Summary screen. |
| | A back arrow to take the user back to the message list. |
| | Up/down arrows in the header bar to allow the user to look at other messages |
| | without returning to the message list. |
| | A trash can icon for deleting the message. |
| 2.6.5 | If the user opens a message containing a Pathology test result, then the tabulated |
| | data will be identical in format to the detail available from the Pathology section |
| | of the Patient summary, see FIG. A44. |
| | If the user touches the “Go to all Pathology results” button, they are transported |
| | to the Pathology summary found on the patient summary screen (not the detail). |
| 2.6.6 | If the user opens a triggered Listening Alert message then the following |
| | additional information/functionality will be shown (an example is shown in |
| | FIGS. A45 and A46): |
| | The parameter that caused the trigger, its value and the date & time the trigger |
| | occurred, |
| | Touching the blue disclosure button beside the information takes the user to the |
| | Listening Alert screen, |
| | Touching the red “Remove listening alert” button reveals a standard confirmation |
| | pop-up where you can confirm or cancel the action |
| 2.6.7 | A maximum of 200 messages will be stored. |
| 2.6.8 | All messages over 7 days old from the current calendar date will automatically be |
| | deleted. |

### 2.7—Patient Charts & Tables

### 2.7.1—Overall Menu

If the user taps on the patient charts tabs the screen
of FIG. A47 is displayed, which allows the
user to navigate to the required chart or data.

### 2.7.2—TPR Chart

| | |
|---|---|
| 2.7.2.1 | The TPR Chart view, see FIGS. A48 (100% view) and A49 (200% view), shall |
| | contain the following information plotted against the date and time that the data |
| | was captured:- |
| | Temperature, |
| | BP (Systolic & Diastolic) Lying or Standing, |
| | Pulse, |
| | Respiratory, |
| | AVPU (Neuro status). |
| 2.7.2.2 | Up to 14 days of observation data can be displayed. |
| 2.7.2.3 | The default view will show the last observation and all the observations that have |
| | occurred in the previous 18 hours. |
| 2.7.2.4 | The screen will allow the user to navigate around it using standard touch-screen |
| | controls, such as, by pinching and expanding to zoom in and out, and by swiping |
| | back and forth to go from one end of the chart to the other. |
| 2.7.2.5 | It shall be possible to tap on any of the lines and symbols on the chart to cause an |
| | observations callout dialogue to be displayed, see FIGS. A50 to A54. This |
| | dialogue will show the following information:- |
| | The date & time of the observation and EWS score, |
| | Temperature value, |
| | BP Systolic, |
| | BP Diastolic, |
| | Pulse, |
| | Respiratory, |
| | AVPU state, |
| | Urine (Localizable option), |
| | The Total EWS score. |
| | Note: Logic for displaying Blood Pressure data labels on graphs is as follows: |
| | a) Lying/standing - if no specific limbs are selected, then capital L/S appears |
| | bracketed above the two top arrow heads; |
| | b) Lying/standing with limb - if a specific limb is also selected, then capital L/S |
| | appears bracketed above the two top arrow heads and the specific limb is |
| | bracketed in lowercase below the bottom arrow heads - either; “la”, “ra”, “ll”, or |
| | “rl”. (Never more than one limb in this scenario); |
| | c) Different limbs - If two different limbs are measured in the same |
| | Observation then either; “la”, “ra”, “ll”, or “rl” appear under each bottom arrow, |
| | but with no bracket. |
| | Logic for the displaying the callout dialogues is as follows: |
| | a) The data labels should be displayed as follows: |
| | Ly sys |
| | Ly dia |
| | St sys |
| | St dia |
| | b) Same as above. |
| | c) Depending on which limbs were used, the order goes left to right, so for |
| | example: |
| | la sys |
| | la dia |
| | ra sys |
| | ra dia |
| | See examples below on FIGS. A51 to A54. FIGS. A53 (100% view) and |
| | A54 (200% view) show how struck through observations will be displayed. |

### 2.7.3—O₂ Saturation Chart

| | |
|---|---|
| 2.7.3.1 | The O2 Saturation chart, see FIGS. A55 (100% view) and A56 (200% view), shall |
| | contain the following information plotted against the date & time that the data was |
| | captured:- |
| | O2 Sat level, |
| | Mask Type, |
| | Flow rate, |
| | The data displayed shall be up to 14 days old. |
| 2.7.3.2 | It shall be possible to tap on any of the plotted points on the chart to cause a temporary |
| | pop-up, shown on FIG. A57, to be displayed showing: |
| | the date & time of the observation; |
| | the mask type; |
| | the recorded O2 saturation level; |
| | the flow rate. |

### 2.7.4—Standard Chart

| | |
|---|---|
| 2.7.4.1 | The Standard chart view shall be as shown in FIG. A58. |
| | The information plotted against the date & time that the data was captured. |
| | The data displayed shall be for the whole admission spell. |
| 2.7.4.2 | If the user doesn't touch the screen for 2 seconds then the navigation options |
| | are removed (see FIG. A58), touching will instantaneously restore them (see FIG. A59). |
| 2.7.4.3 | Considering the navigation options, ‘Part A’ displays standard obs, ‘Part B’ |
| | displays special observations and ‘Struck obs’ displays a table of any struck |
| | through observations. |

(Note: When there is no Part B or Struck Observations to display these options should be removed. Similarly if there is only one page to be displayed then the page counter should also be removed, an example is shown below.)

### 2.7.5—Last Observations Table (Obs Data)

| | |
|---|---|
| 2.7.5.1 | The Obs data view, see FIG. A60, shall contain the |
| | following information shown as a left to right scrollable list: |
| | The time & date of the set of observations, |
| | Temperature and associated EWS score, |
| | BP (Systolic & Diastolic) and associated EWS score, |
| | Pulse and associated EWS score, |
| | Respiratory rate and associated EWS score, |
| | AVPU (Neuro status) and associated EWS score, |
| | O2 Saturation (%), |
| | O2 Flow rate or Delivered O2 conc. (localizable option), |
| | Pain score, |
| | Total EWS score for that set of observations. |
| | Where available the last 14 days observation sets shall be |
| | displayed. Partial Observations will be included in this table. |

### 2.7.6—Special Obs

| | |
|---|---|
| 2.7.6.1 | Sets of special observations collected for the patient are visible on the Patient summary |
| | screen, see FIG. A61. Only the last five Special observations are displayed, summarised |
| | together with the value measured and the date and time they were recorded. Special |
| | observations include:- |
| | Analgesia |
| | Bowels |
| | CVP |
| | Diabetic monitoring |
| | Epidural |
| | Neurological |
| | Pre & Post Nebulizer |
| | Sedation |
| | Urine |
| | Wounds & Drains |
| 2.7.6.3 | For each Special observation the value recorded together with the date & time they were |
| | taken must be displayed. |
| 2.7.6.4 | Where available, special observations must be shown that are up to 14 days old. |

### 2.7.7—EWS Chart

| | |
|---|---|
| 2.7.7.1 | The EWS Chart view shall contain the information shown plotted against the date & time |
| | in FIG. A62. |
| | The data displayed shall be up to 5 days old. |
| 2.7.7.2 | It shall be possible via a separate drop-down list control to jump between this view and the |
| | following charts:- |
| | TPR, |
| | O2 Sats, |
| | Last Observations. |
| 2.7.7.3 | It shall be possible to change the number of days worth of data between 1 and 5 days - this |
| | will be managed via a drop-down list control which shall cause the chart to refresh. |
| 2.7.7.4 | It shall be possible to tap on any of the lines and symbols on the chart to cause an |
| | observations details dialog to be displayed (see FIG. A63), this dialog will give show the |
| | following information:- |
| | The date & time of the observation, |
| | Temperature value BP Systolic, |
| | BP Diastolic, |
| | Pulse rate, |
| | Respiratory rate, |
| | AVPU state, |
| | Colour coded EWS scores for each of the above items, |
| | The Total EWS score. |

(Note that the Observations details pop-up will contain localizable information. This chart should be kept aligned with the Nurse.) It shall be possible (by the use of back and forward buttons) to move between all of the observations that are plotted on the chart.

### 2.8—Investigations

### 2.8.1—Pathology

The user shall be provided with the ability to view Pathology data that has been collected for a particular patient. This data has been organized by data type and date it was collected, see FIG. A64.

| | |
|---|---|
| 2.8.1.1 | The Pathology summary will contain the last 5 |
| | Pathology reports (a report may consist of more |
| | than one test). |
| | Each report heading will be displayed in bold, |
| | followed by the date the data was collected. |
| | Any data that contains data outside of the normal |
| | range will be marked by use of a red asterisk |
| 2.8.1.2 | By tapping on any of the report rows, the user |
| | will be taken to a new screen showing the |
| | details for that set of test results, see FIG. A65. |
| | Each Report will be headed with the date the |
| | sample was collected. |
| | Any samples that contain data that is outside of |
| | the normal range will be highlighted in red. |
| | Each report will end with the Sample number, |
| | the date the sample was received and the date |
| | the sample was collected. |

### 2.8.2—Radiology

The user shall be provided with the ability to view all of the Radiology data that has been collected for a particular patient.

| | |
|---|---|
| 2.8.2.1 | If the Radiology feed has not been validated by |
| | the trust then a warning shall be given to the user |
| | before they are able to see the radiology data to |
| | indicate that it has not been validated. The |
| | validation message shall be removed after the |
| | feed is validated. |
| | The Radiology feature must be able to be enabled/ |
| | disabled by site. |
| 2.8.2.2 | The Radiology view will contain the latest 10 sets |
| | of results. |
| | Each dataset will be identified by its title and date & |
| | time it was collected. |
| | Paging to view each set of results will be as for |
| | Pathology (see section 2.8.1), see FIG. A66. |
| | Tapping on the name of the report will take the |
| | user to the full text report. |
| 2.8.2.3 | An example of the Radiology report layout is shown |
| | in FIG. A67. |

(Note that whilst most data is contained within the report the Report number and the person that ordered the report must be clearly shown at the top of the report.)

### 2.9—Listening Alerts

The user shall be provided with the ability to set a Listening alert for the patient such that should any physiological changes occur then the user will get a notification Message.

(Note: Listening alerts are unique to the user i.e. listening alerts set by other clinicians (even for the same patient) will not show for the current user.

| | |
|---|---|
| 2.9.1 | The Listening alert view will initially (when no listening |
| | services are set) display as shown in FIG. A68. |
| 2.9.2 | On tapping on the ‘Add new listening alert’ button the |
| | dialogue of FIG. A69 will be displayed to allow the user |
| | to select a parameter. The user shall be able to select the one |
| | or more of the following parameters to be alerted on:- |
| | Temperature |
| | Pulse |
| | Systolic BP |
| | Diastolic BP |
| | Respiratory |
| | GCS |
| | O2 Sats |
| 2.9.3 | For each of the parameters selected, the user will be presented |
| | with the current observed value (where it is available) and asked |
| | to enter a high and/or low score around the current value which |
| | will trigger the Listening alert, see FIG. A70. If no High and Low |
| | values are entered then the Listening Alert is not saved. |
| | (Note: The user can choose to set only a high or only a low value.) |
| 2.9.4 | On tapping on the ‘Done’ button, see FIG. A70, the list of |
| | Listening alerts is updated and a yellow listening alert flag will be |
| | shown against the Patient's name in: |
| | The Patient List. |
| | The Patient summary - Alerts, Action & Information list. |
| | On tapping the ‘Done’ button the user is presented with a screen such |
| | as shown in FIG. A71. |
| | (Note: Triggered Listening Alerts should always appear above Set |
| | Listening Alerts.) |
| 2.9.5 | To edit a set “Listening alert” the user must touch the edit button to be |
| | presented with the dialogue of FIG. A72. If the user touches the Edit |
| | Listening Alert button, the user will be presented with the value editing |
| | screen (2.9.2). If the user touches the Remove Listening Alert button, |
| | the Set Alert is removed and the yellow flag removed from the Patient |
| | List and the Patient Summary. |
| 2.9.6 | To remove a Triggered Listening Alert the user can touch the ‘Remove’ |
| | button, to reveal the dialogue of FIG. A73. The user must then tap on |
| | the ‘Remove Listening Alert’ button to confirm the removal. The red |
| | flag is also removed from the Patient List and the Patient Summary. |
| 2.9.7 | When the listening alert is triggered the yellow flag is turned to red both |
| | in the patient list, and the patient summary. See FIG. A74. |
| 2.9.8 | The red triggered flag (also displayed on the patient list) shall remain red |
| | until the listening alert is removed. |
| | (Note: The user cannot edit a triggered listening alert.) |

### 2.10—Miscellaneous

| | |
|---|---|
| 2.10.1 | Refresh functionality is not required for demo release. |
| 2.10.2 | The user shall be able to find out about the application by tapping on the |
| | ‘About’ menu item, see FIG. A75, which shall display the following |
| | information in a dialog box:- |
| | VitalPAC Logo |
| | Name of the application |
| | Build Number |
| | Build Date |
| | TLC Contact info |
| | An active link to the VitalPAC web site |
| | Device Name |
| | Device Model |
| | CE mark |
| 2.10.3 | The About screen shall be accessible only from the More screen (see |
| | section 2.1.5). |

### GLOSSARY

This document uses the following terms and abbreviations which are defined as follows:—

| | |
|---|---|
| AVPU | Alert/Voice/Pain/Unresponsive |
| BMI | Body Mass Index |
| CNS | Central Nervous System |
| CVP | Central Venus Pressure |
| EWS | Early Warning Signs |
| FBC | Full Blood Count |
| FiO2 | Inspired Oxygen |
| GCS | Glasgow Coma Score |
| LBW | Lean Body Weight |
| LOS | Length of Stay |
| Sats | Saturation (Blood/Oxygen Level) |
| SIRS | Systemic Inflammatory Response |
| | Syndrome |
| TLC | The Learning Clinic |
| TPR | Temperate/Pulse/Respiratory |
| VTE | Venus Thrombo-Embolism |

What is claimed is:

1. A system for monitoring the health of a hospital patient, the system comprising:
a score engine configured to identify the status of a patient's health using one or more measurements relating to the patient; and,
a list manager configured to identify a medical practitioner associated with the patient;
wherein the system is configured to send a message to the medical practitioner in dependence on the identified status of the patient's health.

2. The system of claim 1, further comprising:
a remote device being a mobile computing device configured to receive the sent message and notify a user of the remote device of message receipt.

3. The system of claim 1, further comprising:
a measuring device being a mobile computing device configured to receive an input from a user of the device, the input comprising the one or more measurements, the measuring device being further configured to transmit the input; and,
wherein the system is arranged so that the score engine receives the one or more measurements from the input transmitted by the measuring device.

4. The system of claim 3, further comprising:
a server for storing patient information relating to the patient such that it is retrievable, the patient information including the one or more measurements relating to the patient; and,
wherein the system is arranged so that the server receives the input transmitted by the measuring device.

5. The system of claim 1, wherein the score engine calculates a score indicating the status of the patient's health using a risk algorithm which uses at least one of the one or more measurements.

6. The system of claim 5, wherein, the risk algorithm has an AUROC value greater than or equal to 0.80, and preferably has an AUROC value of greater than or equal to 0.88.

7. The system of claim 5, wherein the score engine stores a predefined threshold, and the system sends the message when the value of the score crosses the predefined threshold.

8. The system of claim 1, wherein the list manager stores an association between the patient and the medical practitioner, and wherein the list manager identifies the medical practitioner by identifying the association.

9. The system of claim 1, wherein the list manager stores a patient list for the medical practitioner, the patient list including patients associated with the medical practitioner, wherein the list manager identifies the patient in the patient list to identify that the medical practitioner is associated with the patient.

10. The system of claim 9, further comprising an interface for allowing a user of the system to access the list manager, the interface having a display and being capable of displaying and modifying patient list information.

11. The system of claim 7, wherein content of the message indicates the score value which crossed the threshold and the patient whose measurements were used in calculating the score value.

12. The system of claim 11, wherein the message content indicates the direction which the score crossed the threshold.

13. The system of 11, wherein the message content changes in dependence on the score threshold value.

14. The system of claim 1, wherein the message indicates patient information.

15. The system of claim 2, wherein the remote device receives messages sent to the current user of the remote device, wherein the current user is identified as the user logged into the remote device.

16. The system of claim 2, wherein the remote device is a notification device configured to provide the user with instructions for obtaining content of the message.

17. The system of claim 2, wherein the remote device is an analysis device configured to provide the user with the message content.

18. The system of claim 17, wherein the analysis device is additionally configured to provide the user with patient information, to assist the user in administering the correct treatment to the patient.

19. The system of claim 17, wherein the analysis device is additionally configured to receive data from the current user and transmit the data.

20. The system of claim 3, wherein the measuring device is configured to receive the input from the user via a graphical user interface of the measuring device.

21. The system of claim 3, wherein the input additionally comprises administrative information relating to the patient.

22. The system of claim 3, wherein the measuring device notifies a current user of the measuring device when the one or more measurements need to be taken from the patient, or how long overdue are those one or more measurements, wherein the measuring device identifies the current user as the user logged-in to the measuring device.

23. The system of claim 3, wherein the system is configured so that the measuring device receives a notification in response to transmitting the input, the notification indicating whether or not someone has taken responsibility for progressing the care of the patient.

24. The system of claim 2, further comprising a base-station configured to communicate wirelessly with the remote device and the measuring device, the base-station including at least one of the score engine and the list manager.

25. The system according to claim 1, further comprising a base-station configured to communicate wirelessly with the remote device and the measuring device, the base-station including the list manager, and wherein the measuring device includes the score engine.

26. The system according to claim 1, wherein identifying the status of the patient's health comprises identifying changing health of the patient; and,
sending a message to the medical practitioner in dependence on the identified status of the patient's health comprises sending a message to the medical practitioner if changing health of the patient is identified.

27. A computer implemented method for monitoring the health of a hospital patient, the method comprising:
a. electronically identifying the status of a patient's health, using one or more measurements relating to the patient; and,
b. electronically identifying a medical practitioner associated with the patient and sending an electronic message to the medical practitioner, in dependence on the identified status of the patient's health.

28. The computer implemented method of claim 27, further comprising:
receiving the sent electronic message at a remote device, the remote device being a mobile computing device; and,
notifying the medical practitioner of message receipt, the medical practitioner being a user of the remote device.

29. The computer implemented method of claim 27, further comprising:
electronically receiving the one or more measurements at a measuring device, the measuring device being a mobile computing device.

30. The computer implemented method of claim 27, further comprising
electronically storing patient information relating to the patient such that it is retrievable, the patient information including the one or more measurements relating to the patient.

31. The computer implemented method of claim 29, further comprising:
electronically receiving the administrative information at the measuring device with the one or more measurements.

32. The computer implemented method of claim 27, wherein step a. comprises:
electronically calculating a score indicating the status of the patient's health using a risk algorithm which uses at least one of the one or more measurements.

33. The computer implemented method of claim 32, wherein the risk algorithm has an AUROC value greater than or equal to 0.80, and preferably has an AUROC value of greater than or equal to 0.88.

34. The computer implemented method of claim 32, further comprising:
electronically storing a predefined threshold; and,
wherein step b. comprises, sending the electronic message when the value of the score crosses the predefined threshold.

35. The computer implemented method of claim 27, further comprising:
electronically storing an association between the patient and the medical practitioner; and,
wherein electronically identifying a medical practitioner associated with the patient comprises identifying the electronically stored association.

36. The computer implemented method of claim 27, further comprising:
electronically storing a patient list for the medical practitioner, the patient list including patients under the responsibility of the medical practitioner; and,
wherein electronically identifying a medical practitioner associated with the patient comprises electronically identifying the patient in the patient list.

37. The computer implemented method of claim 27, further comprising:
setting the content of the electronic message to indicate the score value which crossed the threshold and the patient whose measurements were used in calculating the score value.

38. The computer implemented method of claim 37, further comprising:
setting the content of the electronic message to indicate the direction which the score crossed the threshold.

39. The computer implemented method of claim 37, further comprising:
changing the content of the electronic message in dependence on the score threshold value.

40. The computer implemented method of claim 27, further comprising:
setting the content of the electronic message to indicate patient information.

41. The computer implemented method of claim 28, further comprising:
providing the medical practitioner with electronic instructions for obtaining content of the electronic message, at the remote device.

42. The computer implemented method of claim 41, wherein the electronic instructions include: a telephone number, a patient identifier, a patient location.

43. The computer implemented method of claim 28, further comprising:
providing the medical practitioner with the content of the electronic message, at the remote device.

44. The computer implemented method of claim 43, further comprising:
providing the medical practitioner with electronic patient information, at the remote device, to assist the medical practitioner in administering the correct treatment to the patient.

45. The computer implemented method of claim 44, wherein the patient information includes at least one of the following: patient medical records, patient test results, patient details, patient location.

46. The computer implemented method of claim 43, further comprising:
electronically receiving an input from the medical practitioner, at the remote device.

47. The computer implemented method of claim 29, further comprising:
electronically receiving an input from a user, at the measuring device, the input comprising the one or more measurements relating to the patient.

48. The computer implemented method of claim 47, further comprising:
electronically notifying the user when the one or more measurements need to be taken from the patient, or how long overdue are those one or more measurements.

49. The computer implemented method of claim 29, further comprising:
electronically receiving, at the measuring device, an indication of whether or not someone has taken responsibility for progressing the care of the patient.

50. The computer implemented method according to claim 27, wherein electronically identifying the status of the patient's health comprises electronically identifying changing health of the patient; and,
electronically identifying a medical practitioner associated with the patient and sending an electronic message to the medical practitioner, in dependence on the identified status of the patient's health comprises electronically identifying a medical practitioner associated with the patient and sending an electronic message to the medical practitioner, if changing health of the patient is identified.
